(19) United States
(12) Patent Application Publication
(10) Pub. No.: US 2007/0178503 A1
(43) Pub. Date: Aug. 2, 2007

(54) In-situ genomic DNA chip for detection of cancer

(76) Inventor: Feng Jiang, Ellicott City, MD (US)

Correspondence Address: DLA PIPER US LLP;ATTN: PATENT GROUP, 1200 NINETEENTH STREET, NW, WASHINGTON, DC, 20036, US

(21) Appl. No.: 11/641,008

(22) Filed: Dec. 19, 2006

Related U.S. Application Data
(60) Provisional application No. 60/751,205, filed on Dec. 19, 2005.

(51) Int. Cl. C12Q 1/68; C12M 1/34

(52) U.S. Cl. 435/6; 435/287.2

(57) ABSTRACT

An in situ genomic DNA chip can be used directly on clinical specimens in situ for detecting, diagnosing and/or predicting diseases, especially diseases characterized by a genetic aberration such as cancers, by simultaneous detection of one or more unique genetic aberrations using one or more multiple specific probes. The present invention offers high sensitivity for detection of such genetic aberrations and, further, has substantial implications for large-scale population-based molecular epidemiologic studies and therapeutic interventions.

This application claims priority from U.S. Provisional Application Ser. No. 60/751,205 filed Dec. 19, 2005. The entirety of that provisional application is incorporated herein by reference.

This invention was made with government support under Grant No. CA113707 awarded by the National Institutes of Health. The Government may have certain rights in this invention.

### FIELD

The present invention relates generally to detection and diagnosis of diseases, and in particular, to an in situ genomic DNA array for detection of a disease with genetic aberrations, such as lung cancer. The present invention offers high sensitivity for cancer detection and has substantial implications for large-scale population-based molecular epidemiologic studies and therapeutic interventions.

### BACKGROUND

Lung cancer is among the leading cancer killer in both men and women. In the United States, approximately 170,000 new lung cancer cases are diagnosed each year, and less than 15% of patients survive to 5 years after diagnosis (1). The prognosis for patients with lung cancer correlates strongly with the stage of the disease at the time of diagnosis. More than two thirds of patients with lung cancer have regional lymph node involvement or distant metastasis at the time of presentation (2). The unsatisfactory cure rate and poor prognosis in these patients has led the NCI to designate technology development and biomarker discovery for the early detection of lung cancer a high research priority.

A number of molecular genetic approaches have been developed to detect cancer cells in various types of specimens. For example, different polymerase chain reaction (PCR)-based assays, including microsatellite, mutation, and methylation analyses, have been evaluated extensively in the diagnosis of cancer. However, these techniques do not address the technical issues specific to the detection of cancer cells in situ and, thus, cannot be used directly on clinical specimens. Therefore, the data obtained from such assays do not represent sample heterogeneity or detect small populations of abnormal cells that may have characteristics indicating the initiation or progression of cancer. Although microarray analysis of more than 10,000 genes simultaneously is promising for the identification of critical genes underlying cancer progression, it is limited in its reproducibility, accuracy, and cost-effectiveness and is labor intensive. In addition, although antigen-based methods, such as immunohistochemistry for identifying proteins, can be performed in situ, they can only detect a single antibody at a time and have excessively low sensitivity.

U.S. Pat. No. 6,797,471 to Katz et al., incorporated by reference herein in its entirety, claims genetic tools for identifying a subject at risk for the development of non-small cell lung cancer.

However, none of these techniques have been shown to be practical or universally applicable in a clinical setting.

### SUMMARY

One aspect of the present invention relates to an in situ genomic DNA array for detection of a disease with genetic aberrations, said DNA array comprising a substrate, and a plurality of genomic DNA probes immobilized to said substrate, wherein said plurality of genomic DNA probes are selected to identify a genetic signature of said disease and are capable of interphase multiple fluorescence in situ hybridization (FISH) with a tissue or cell sample.

Another aspect of the present invention relates to a method for detecting a disease characterized by at least one, two, three or four genetic aberrations, said method comprising: incubating a tissue or cell sample with an in situ genomic DNA array comprising a plurality of genomic DNA probes selected to identify a genetic signature of said disease and capable of interphase multiple fluorescence in situ hybridization (FISH) with said tissue or cell sample, detecting a signal from said in situ genomic DNA array, and (c producing a diagnosis based on the signal detected from said in situ genomic DNA array.

In yet another aspect the present invention relates to a kit comprising at least one genetic probe, wherein the genetic probe comprises a nucleotide sequence specific to a gene having a genetic aberration in a diseased (abnormal) state, a chromosomal centromeric probe consisting essentially of a nucleotide sequence of the chromosome centromere corresponding to the gene, wherein said genetic probe and said chromosomal centromeric probe each further comprise a fluorescent label, and a suitable container.

Another aspect the present invention relates to a kit comprising at least four genetic probes, wherein each of the genetic probes comprise a nucleotide sequence specific to a first, second, third, and fourth gene, each having a genetic aberration in a diseased (abnormal) state, a chromosomal centromeric probe, each consisting essentially of a nucleotide sequence of the chromosome centromere corresponding separately to the first, second, third, and fourth gene, wherein said genetic probes and said chromosomal centromeric probes each further comprise a fluorescent label, and a suitable container.

### BRIEF DESCRIPTION OF THE FIGURES

FIG. 1 shows morphologic and molecular genetic aberrations during lung cancer progression. Preneoplastic cells contain several molecular genetic abnormalities identical to some of the abnormalities found in overt lung cancer cells.

FIG. 2 shows genomic signatures in primary NSCLC. Vertical red bars represent the separation of chromosomes. Genes that increased in copy number (normalized intensity ratios >0) are as indicated above the x-axis, and those that decreased in copy number (ratios <0) are as indicated below the x-axis (3).

FIG. 3 shows M-FISH analysis of a lung TMA. Left tumor tissue element showing clear, bright signals: p16 (green), SFTPA (gold), hTERT (blue), and GC20 (red).

FIG. 4 shows FISH analysis of a lung cancer specimen with the GC20-specific probe (green) and chromosome 3 probe (red) used as a control. Left, there are fewer green signals than red signals, indicating deletion of GC20. Right, GC20 deletion occurred in both adjacent bronchial and carcinoma cells.

FIG. 5A shows CGH analysis of a lung tumor demonstrating a high-level copy-number gain at 5p13 (5A). Tumor DNA was labeled with green fluorochrome, and normal DNA was labeled with red fluorochrome.

FIG. 5B shows the ordering of 29 BACs used as probes in FISH mapped on 5p13.2. The middle scale is a result of the DNA copy number in a lung cancer cell line, indicating the smallest overlapping region with maximal amplification containing six genes.

FIG. 5C is an RT-PCR analysis showing that most of the cancer cell lines have a high level of transcription of the SKP2 gene. β-Actin was used as the control.

FIG. 5D is a FISH analysis of lung cancer specimens showing increased copies of green probes, indicating amplification of the SKP2 gene.

FIG. 6 shows effect of SKP2 depletion on cyclin E/CDK2 expression and centrosome amplification. (A) Lung cancer cells were transfected with siRNAs against SKP2 and subjected to Western blot analysis. (B) The cells were examined with anti-a-tubulin (green) or -λ-tubulin antibodies (red), suggesting that depletion of SKP2 in cancer cells leads to inhibition of centrosome amplification. (C) Results from three independent experiments.

FIG. 7 is a Kaplan-Meier curve comparing patients with >10 SP-A deletions to those with ≦SP-A deletions.

FIG. 8A is a achematic p16 gene map detected by a gene-specific probe developed with our novel technique. Top, genomic structure of p16. The four black bars denote the size and location of LD-PCR products, which are confirmed by genomic fiber detected by fiber FISH. Bottom, the green fiber shows the product of DOP-PCR, which is a 27 kb gene FISH probe spanning the entire p16.

FIG. 8B shows the commercially available p16 FISH probe, LSI p16, is 190 kb in length and contains not only p16 (INK4A) but also several genetic loci, including D9S 1749, D9S 1747, p14 (ARF), D9S 1748, p1 5 (INK4B), and D9S 1752.

FIG. 8C shows a metaphase spread of a normal human lymphocytic cell hybridized with the probe showing the chromosomal location of the p16-specific probe. The green signals (arrows) indicate that the location of the probe is on chromosome 9p21.

FIG. 8D shows dual-color FISH analysis of a paraffin embedded lung cancer section with the p16-specific probe (green) and chromosome 9 centromeric probe (red). Most of the cells contain fewer green signals than red ones, indicating deletion of p16 (arrows).

FIG. 9A is a picture of BAL sample showing unremarkable bronchial epithelium on cytospin preparation (60×).

FIG. 9B is a picture of the bronchial brushings in 9A showing a normal diploid population on an M-FISH assay (600×).

FIG. 9C is a picture of a BAL sample from the tumor-bearing side showing apparently abnormal ciliated bronchial epithelial cells.

FIG. 9D is a picture of a M-FISH assay showing genetic aberrations in the cells from the sample in FIG. 9C showing multiple copies of each probe (1000×).

FIG. 10 is a schematic showing automatic dot-counting multiple color signals using a Cytovision Automated workstation to examine bronchial brushings with LAVysion consisting of one centromeric probe (CEP6) labeled with aqua, and three locus-specific probes for 5p15.2, 7p12 (EGFR), 8q24.12-q24.13 (C-MYC) labeled with green, red, and gold, respectively.

FIG. 11 is a schematic showing an in situ genomic DNA assay. The template DNA of the genes of interest was labeled with four fluorochromes, and the probes were arranged in four elements. Each element included a cocktail of four probes and then printed on a coated glass coverslip. After the hybridization solution was added to the slide, the coverslip was inverted over the slide. Four corresponding squares were marked on the back of the slide, according to the location of the four elements on the coverslip. After hybridization and post-washing, signals were counted and images captured, based on the marked line on the back side of the slide.

FIG. 12 shows a strategy for labeling the specific DNA probes with four fluorochromes. A. All current available fluorochromes (DAPI, DEAC, FITC, Cy3, Cy3.5, Cy5, Cy5.5, Cy7, and LaserPro IR 790) and their fluorescence excitation of spectra. B. Only four fluorochromes will be used in the current study, because there is no overlap of wavelengths among them. The signals will be distinguished clearly from each other under appropriate filters.

FIG. 13 shows validation of the gene-specific genomic probes on the chromosomal spreads of normal lymphocytes and normal bronchial epithelial cells and of their corresponding chromosomal centromeric probes used as controls. A, Chromosomal spread of normal metaphases shows the ENO1 probe on 1p36.23 (yellow), the CEPI probe on the centromere of chromosome 1 (red), the p16 probe on 9p21.3 (green), and the CEP9 probe (aqua). B, Normal interphases show two yellow signals of the ENO1 probe, two red signals of CEP1 probe, two green signals of the CEP10, and two aqua signals of the CEP9 probe.

FIG. 14 is a schematic p16 gene map of normal DNA fiber detected by a gene-specific probe developed with the LD-DOP-PCR strategy. Top, genomic structure of p16. The four black bars denote the size and location of LD-PCR products, which were confirmed by the genomic fiber detected by FISH. Bottom, the green fiber shows the product of DOP-PCR, which is a 27-kb gene FISH probe spanning the entire length of p16.

FIG. 15 shows representative hybridization images from an assay using an in situ DNA chip having a one-color fluorescence-labeled probe in each element (square) tested in the specimen. Square A shows only a red signal, square B (in all the tested samples) only green signals, and so on, indicating that there was no mixture of the probes between the different squares during the hybridizations.

FIG. 16 is representative hybridization images of LAVysion-negative and in situ DNA chip-positive cancer cells. A, Bronchial washing sample showing an unremarkable bronchial epithelium in a cytospin preparation (original magnification×60). B, Results of a LAVysion assay of the bronchial brushing in A showing a normal diploid population (original magnification×600). C, In situ DNA chip assay results showing genetic aberration.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

One aspect of the present invention is directed to an in situ genomic DNA array that simultaneously measures the tumor-specific genetic changes in easily accessible surrogate specimens and provides early tumor diagnosis. In one embodiment, the present invention is directed to an in situ genomic DNA array for the detection of lung cancer. Genomic signatures in primary lung cancer have been defined and genomic probes for these signatures have been developed.

The genetic signatures that are directly associated with early progression of lung cancer constitute biomarkers in early lung cancer diagnosis. However, lung cancer is a heterogeneous disease; a single biomarker is not sufficient to predict a clinically significant phenotype with acceptable accuracy. By taking advantage of multifluorochrome probe labeling and detection methods with a cocktail of highly specific probes for simultaneous, comprehensive detection of unique genetic aberrations, the in situ strategy offers high sensitivity and specificity for early lung cancer detection despite the heterogeneity of lung tumorigenesis.

Lung cancer is the end stage of multistep carcinogenesis, featuring field defects in the airway of smokers. There is concordance of the degree of genetic aberrations in all target tissues, such as in respiratory epithelial cells exfoliated from sputum and oral brushings and the lung cancer tissue exposed to tobacco carcinogens. Sputum and oral brushings may be used as surrogate tissues for assessing carcinogenic lung damage.

The in situ array of the present invention is suitable for automated image analysis for dot counting with an automatic procedure, which would provide fast quantitative and qualitative assessment of the multiple biomarkers.

Specifically, the present invention (1) identifies a set of specific genetic probes that provide the greatest overall sensitivity and specificity for detection of lung cancer cells, (2) provides an in situ genomic DNA array consisting of the most clinicopathologically relevant probes, and (3) provides an automated genetic test using the in situ genomic DNA array and automatic fluorescent dot counting.

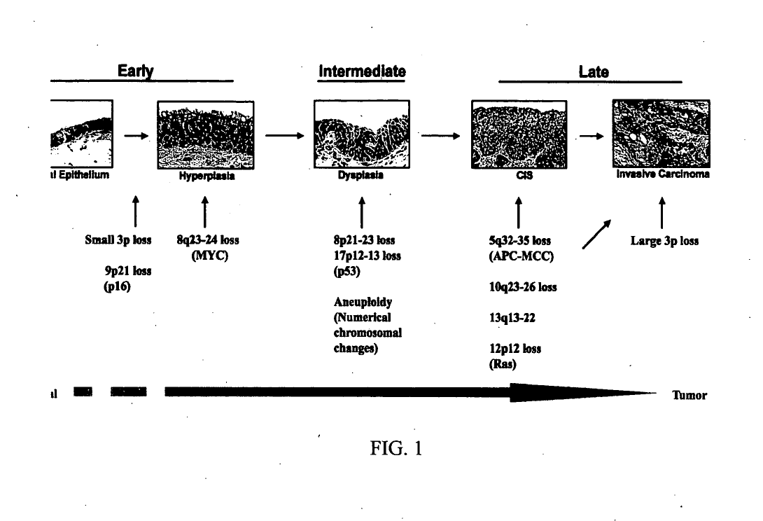

During lung cancer initiation and progression, the genetic alterations accompany oncogenic transformation in a preferred order at several 3p sites followed by 9p ( p16 locus), with the earliest changes occurring in histologically normal epithelium (6-8). These changes are followed by changes at 8p, 17p, 5q, and 10q upregulation of hTERT, myc, ras, bcl-2, and cyclin D1; and p53 mutations (9) (FIG. 1). These observations are consistent with the multistep model of carcinogenesis and field cancerization, in which the whole tissue region that is repeatedly exposed to tobacco smoke is susceptible to development of multiple separate, clonally unrelated foci of neoplasia (6). Therefore, no single biomarker is sufficient to predict a clinically significant phenotype with acceptable accuracy (10). Efforts have been directed toward developing molecular techniques for simultaneously measuring a panel of biomarkers rather than a single or few biomarkers for early detection of lung cancer.

Development of polymerase chain reaction (PCR)-based molecular genetic analyses, and high-throughput arrays including cDNA and protein microarrays, have been investigated for their potential in risk assessment of lung tumors (11-20). However, although they are powerful, the available analysis tools do not adequately address technical issues specific to the diagnosis of cancer cells in vitro, such as a limited cell number, sample heterogeneity, and cost-effectiveness (21). Furthermore, these methods cannot be performed in situ and directly applied to clinical specimens. Therefore, none of them have proven to be practically and universally applicable in a clinical setting for early detection of lung cancer (21).

Although Northern blotting and cDNA array and immunohistologic analysis have been applied for measuring gross upregulation and downregulation of genes, the identification of consistent genomic alterations, such as gene amplification, deletion, and translocation in cancer specimens has been proven to provide important diagnostic and predictive information (22-24). A recent study confirmed a major direct role for DNA copy-number alterations in the transcriptional program of human tumors, suggesting that such alterations may contribute to the development or progression of cancer (23). Furthermore, it is now clear that amplification and/or overexpression of c-erb-B-2 correlates with an adverse outcome in breast carcinoma; gene amplification is a better predictor of outcome than overexpression is (25). In addition, discordance between the gene copy-number and gene expression has also been described in several oncogenes and tumor suppressor genes (TSGs) other than c-erb-B-2, such as c-myc, RB1, and p53. Such discordance may necessitate gene copy-number analysis rather than immunohistology (25). The recently designed M-FISH technique reveals cell-to-cell heterogeneity, enables detection of minor subpopulations of genetically distinct cells, and allows for direct visualization and examination of chromosomal and large genomic aberrations at the same time. Also M-FISH does not require microdissection or DNA, RNA, or protein preparations (26-27). Importantly, this technique is directly applicable to cellular material with the use of a small number of cells. Furthermore, when compared with the above-described high-throughput techniques, it is more quantitative and less laborious and requires fewer samples. It have been proved that M-FISH works particularly well with clinical specimens (27-28). Therefore, the present invention by using an in situ genomic DNA array with multiple specific probes for simultaneous, comprehensive detection of unique genetic aberrations provides the most practical, cost-effective method for early detection of lung carcinogenesis.

A major obstacle to early detection of lung cancer has been selection of biologic materials for assessing end points. Although bronchial brushings and lavage specimens can provide bronchial epithelial cells from distinct areas of the airway for various morphologic and biomarker examinations, collection of these specimens is expensive and invasive, therefore, they are not routinely obtained from individuals having only a moderate risk of lung cancer. Slaughter introduced the concept of field cancerization, which was supported by the fact that most of the epithelial surface of the aerodigestive tract is likely to be exposed to many of the common carcinogens and therefore at increased risks for both lung and head and neck cancer (29-30).

Epithelium in the respiratory tract exposed to carcinogens contains clonal genetic alterations that are important in early lung tumorigenesis and may contribute to an increased risk of cancer in smokers. There may be concordance in the degree of genetic aberrations among all target tissues, such as respiratory epithelial cells exfoliated from sputum, oral, and bronchial brushings. Belinsky et al. (31) reported that detection of p16 and MGMT CpG hypermethylation in the sputum could be useful in predicting lung cancers. Recently, using a four-color FISH probe targeting centromere 6, 5p15.2, 7p12, and 8q24, Romeo et al. (27) found a high number of abnormal cells in all tumor and sputum specimens obtained from patients with lung cancer. Although the sensitivity and specificity of the M-FISH probe set must be validated in further controlled clinical trials, this study clearly demonstrates that sputum may be used as surrogate targeted tissue for in situ hybridization analysis with multiple genomic probes. Furthermore, Ayoub showed a perfect correlation of expression of retinoic acid receptor- 0 between palatal and bronchial brushings (32). Mao et al. determined the DNA hypermethylation status of p16, death-associated protein kinase, and GSTP1 in paired bronchial and buccal brushings obtained from former smokers (33) and implied that the frequency of hypermethylation was similar in the paired samples. These reports support the usefulness of buccal brushings as surrogate tissue for detection of lung cancer. Since epithelial cells exfoliated from sputum, oral brushings and lung cancer tissue may share many common genetic abnormalities that may constitute targets for cancer detection, these specimens may be used as surrogate materials in evaluating carcinogenic damage with our in situ genomic DNA array.

Early-detection assays provide relatively fast quantitative and qualitative determination of biomarkers. All of the current molecular techniques are very time consuming, and several issues, especially quality, validation, and interpretation, remain to be resolved before these assays can be used as diagnostic tools for routine work (21-34). One of the most important applications of in situ hybridization is dot counting, which makes quantitative and qualitative determination of biomarkers possible (25-26). To eliminate analytical errors common in manual testing and reduce the labor expended and time consumed, automated image analysis has been expedited for in situ hybridization dot-counting analysis. Netten et al. combined in situ hybridization with an automated fluorescence microscopy system that can examine 500 cells in approximately 15 minutes to determine the number of signals in each cell nucleus (35). The inventors believe that the CytoVision SPOT AX automated workstation (Applied Imaging) can greatly reduce the time required for dot counting from 120 minutes with manual counting to 10 minutes per slide in counting 200 cells in the same specimens, with accuracy similar to that of the manual method. Because one of the aspects of the present invention is designed and developed on the basis of in situ hybridization, the application of the automated dot-counting system will make the in situ genomic DNA array a powerful tool for early lung cancer detection and risk assessment in clinical settings.

Various embodiments of the present invention demonstrate the following advantages:
1. Establishment of the first panel of genomic biomarkers to detect molecular changes in lung cancer and improvement of understanding of early lung carcinogenesis.
2. Development of a novel diagnostic assay with high sensitivity and specificity for early detection of lung cancer.
3. Use of easily accessible tissues as surrogate materials for early lung cancer detection.
4. Substantial implications for future large-scale, population-based molecular epidemiologic studies and clinical applications. Identification of surrogate biomarkers that best predict genetic alterations would be extremely useful for identifying subjects at particularly high risk for clinically apparent lung cancer. Such subjects could be targeted for enrollment in screening trials using spiral computed tomography (CT). Moreover, genetic markers may be relevant for adjunctive screening of subjects found to have changes on spiral CT scans. The array will have extensive clinical implications, including staging and postoperative surveillance of lung cancer as well as chemopreventive interventions.
5. Risk assessment for early diagnosis of, and staging of other types of cancer when combined with other appropriate tumor-specific genes. For example, development of an in situ genomic DNA assay consisting of biomarkers for the genetic alterations underlying the progression of urinary bladder cancer will provide a promising approach for noninvasive detection of bladder cancer in urine specimens.

### EXAMPLES

### Example 1

Genetic Signatures and Their Significance in Lung Tumorigenesis

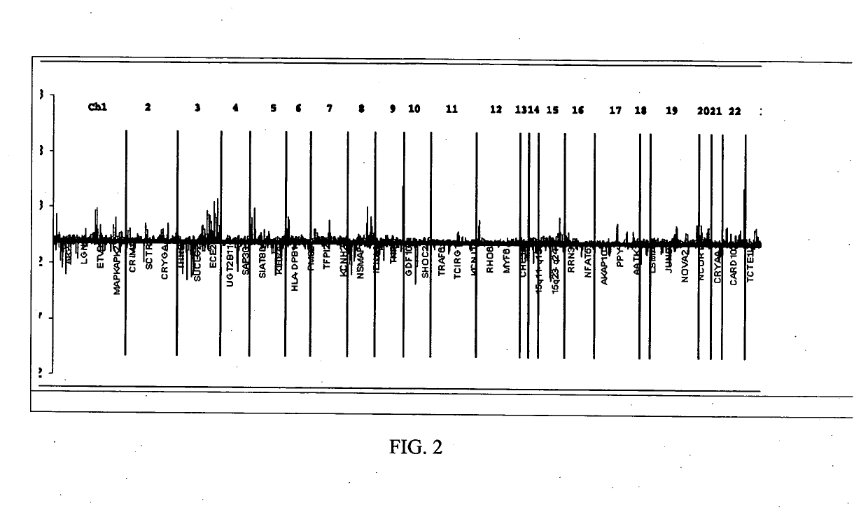

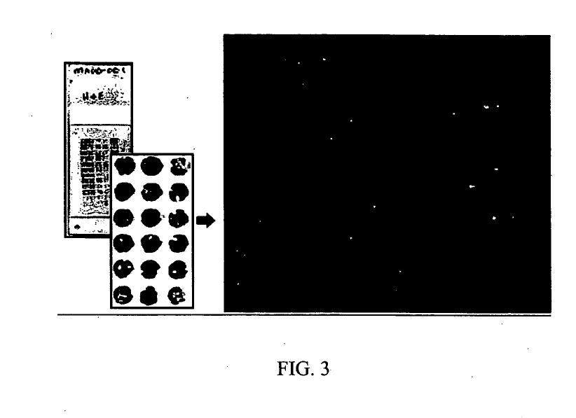

Characterization of genomic aberrations in human tumors has greatly enhanced the identification of tumor-relevant genes (genetic signatures), which will be developed as new diagnostic markers for early detection of cancer. We have worked in determining the role of critical genes in carcinogenesis, including that of bladder, renal, lung, and cervical cancers, using molecular and cytogenetic tools (36-42). Recently, we characterized in detail the genomic copy-number changes associated with individual genes in primary NSCLCs by performing high-resolution comparative genomic hybridization onto a cDNA microarray (3). We found that primary NSCLCs shared common frequent distribution of recurrent aberrations of sets of genes (FIG. 2 and Table 1). We also precisely defined the genomic difference between the two most common lung tumor subtypes, squamous cell carcinoma and adenocarcinoma, and generated a clear genomic profile of primary adenocarcinoma, which showed distinct difference between the tumors and their paired normal lung tissues due to a cluster of genomic aberrations. To investigate whether the findings can be used as molecular markers for early detection of lung cancer, we developed a panel of gene-specific probes for the genes p16, SFTPA1, hTERT, and GC20 and then tested the probes on lung tissue microarray (TMA) slides. We demonstrated that an M-FISH assay with the probes worked well on paraffin-embedded tissue specimens and reliably detected rearrangement of targeted genes (FIG. 3). The results also confirm that changes in the genomic copy number of these genes are early events in lung tumorigenesis (3).

TABLE 1
The genes that show genomic copy number aberrations in
primary NSCLC specimens by comparative genomic
hybridization (CGH) analysis of cDNA microarrays

| Gene | Loc. | Gene | Loc. | Gene | Loc. | Gene | Loc. | Gene | Loc. |
|---|---|---|---|---|---|---|---|---|---|
| FCN3 | 1p35 | SH3BP | 3p24 | SKP2 | 5p13 | ARHGEF | 13q33 | TGFBR | 3p22 |
| MACF1 | 1p32 | GC20 | 3p21 | SFTPC | 8p21 | MADH6 | 15g21 | GNB2L | 5q35 |
| CRABP | 1q21 | POLR | 3q28 | YWHAZ | 8q23.1 | NME2 | 17q21 | FGF4 | 11q13 |
| SNRPE | 1q32 | PLOD | 3q23 | LAPTM | 8q22.1 | RPL38 | 17q23 | MAGED | Xp11 |
| JTB | 1q21 | GPX3 | 5q23 | P16 | 9p21 | EPB41L | 18p11 | HYAL2 | 3p21 |
| Cks1 | 1q21 | DUSP | 5q34 | ELAVL | 9p21 | USP14 | 18p11 | hTERT | 5p15 |
| TNA | 3p22 | CD74 | 5q32 | ACTA1 | 10q23 | RPS16 | 19q13 | GPC3 | 11q13 |
| GNAI2 | 3p21 | CCT5 | 5p15 | SFTPA | 10q22 | RPS21 | 20q13 | RPL37 | 5p13 |

As described below, we primarily characterized and confirmed several of the signatures that are useful for the early detection of lung cancer.

1). Potential TSG at Chromosome 3p21.3 in Tobacco-Related Lung Cancer

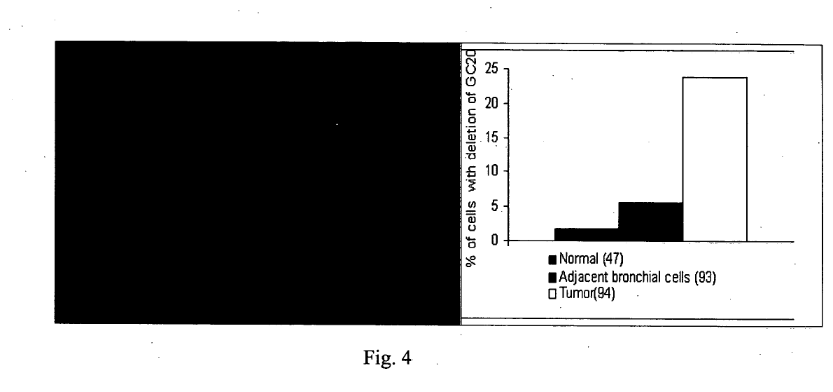

Recurrent homozygous loss of 3p21.3 is one of the most frequent and earliest acquired genetic aberrations in the multistage pathogenesis of tobacco-related lung cancer. Strong evidence suggests that this region contains TSGs, which are likely to play a causative role in lung cancer progression (43). GC20 is located within the region (Table 1). In a retrospective FISH study of microdissected lung carcinomas and adjacent normal bronchial epithelium obtained from 96 patients with stage I lung cancer, we demonstrated that deletion of GC20 occurs in both adjacent bronchial cells (5.57±5.21%) and, to a much higher degree, carcinoma cells (23.79±18.78%). Additionally, this deletion occurred more frequently in tumors in smokers than in nonsmokers (p<0.001). These findings suggest that inactivation of this region is an early event in smoking-related lung tumorigenesis and important in its initiation (FIG. 4) (4). This implies that a GC20 specific genetic probe can be used as a biomarker for early detection of lung tumors.

2). S-Phase Kinase-Interacting Protein 2 (SKP2) at the 5p13.2 Amplicon

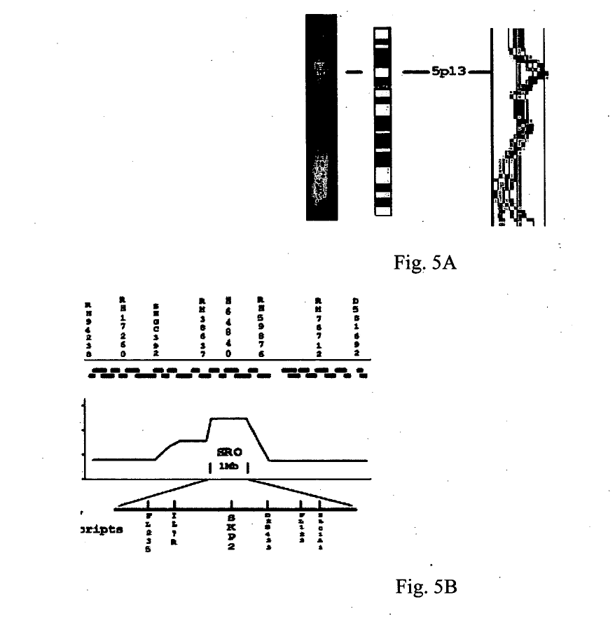

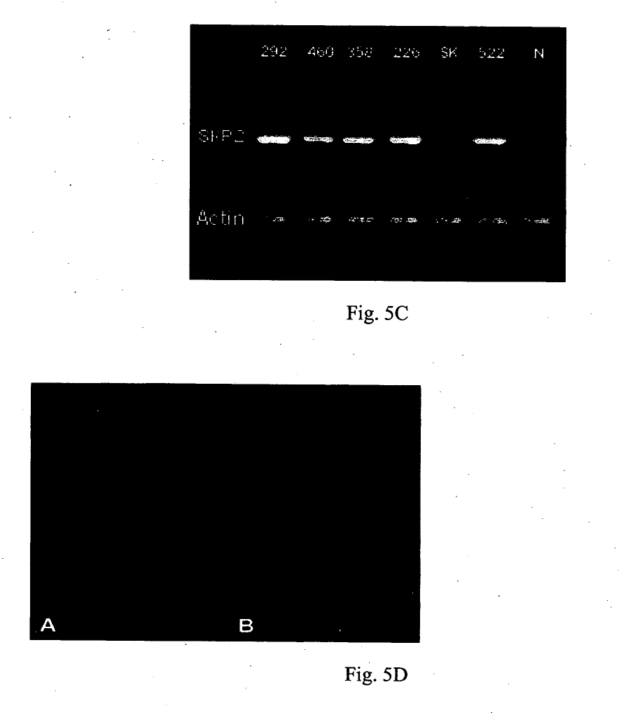

Amplification of DNA in certain chromosomal regions allows for activation of critical genes involved in the development of tumors. Considerable efforts have been made to explore amplified regions and genes in lung tumorigenesis. Several genes, such as MYC at 8q24, EGFR at 7q32, and CCND1 at 11q13, have been successfully identified in this manner and shown to be associated with a malignant phenotype of lung tumorigenesis. However, cumulative results of recent CGH studies, including our own cDNA microarray CGH analysis, demonstrate that additional amplification targets, including 5p13.2, have yet to be identified (FIGS. 5A and B). We constructed a bacterial artificial chromosome (BAC) covering the amplified region, defined a relatively smaller amplified region of overlap (1 Mb), and identified the SKP2 gene as a strong candidate target for amplification, because there is a positive correlation between 5p13.2 amplification, an increased relative copy number of SKP2, and the transcription level in lung cancer cell lines (FIGS. 5C and D).

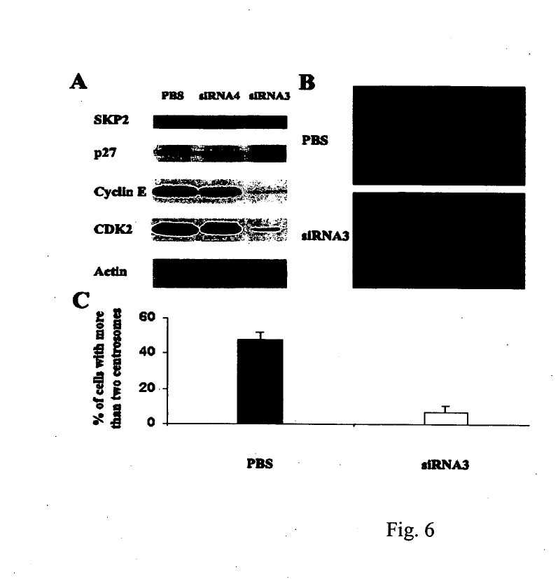

SKP2 displays a promotion function in the cell cycle and is implicated in ubiquitin-mediated degradation of several key regulators of mammalian G1 progression (44). We further inhibited the expression of SKP2 in lung cancer cells by transfecting them with small interfering RNAs (siRNAs) targeted against SKP2 and showed SKP2-siRNAs specifically and efficiently reduced levels of SKP2 protein. Cell proliferation was reduced by 12%, and apoptosis was increased by 36%. Furthermore, 36% of the cells accumulated in the sub-G1 phase, and the population of cells in the G1 phase decreased to 37%. In addition, the SKP2-depleted cells showed decreased levels of cell cyclin-dependent kinase 2 and cyclin E. Correspondingly, only 7% of the treated cells had multiple centrosomes, compared with 46% of the control cells, suggesting that a reduced level of SKP2 in cancer cells leads to inhibition of centrosomal amplification and spindle abnormalities, which may result in failed cytokinesis and chromosome loss or missegregation (FIG. 6). Our data not only confirmed the importance of p27 in lung tumorigenesis but also demonstrated that a reduced level of SKP2 expression in cancer cells causes downregulation of cyclin E/CDK2 and implied an oncogenic role for SKP2 in centrosome abnormalities that are critical in causing genetic instability in tumor cells. These results imply that SKP2 plays an oncogenic role in lung tumorigenesis and that SKP2 biomarkers may be used for monitoring the initiation and progression of lung cancer and response to therapy (45).

3). Prognostic Value of Surfactant Protein A (SP-A) Deletions at 10q22-23 in Tobacco-Related Lung Cancer

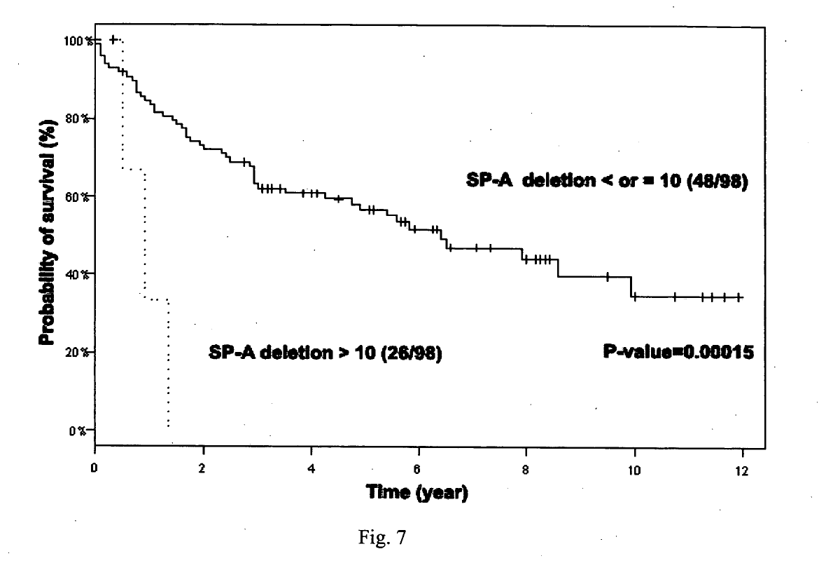

The SP-A gene locus localizes at chromosome 10q22-23, one of the most common genomic imbalance regions in lung cancer. We determined the role of SP-A in tumor progression and its prognostic value by evaluating paraffin-embedded tissue samples obtained from 96 stage I NSCLC specimens with microdissection techniques and FISH with a specific DNA probe. SP-A was deleted in carcinoma tissues (87%) and adjacent normal appearing bronchial tissues (32%). These deletions were more common in the specimens obtained from the patients who smoked than in those obtained from patients who had never smoked. There was a statistically significant correlation between SP-A deletions in adjacent normal-appearing bronchial tissues and those in tumors. SP-A deletions in tumor and adjacent normal-appearing bronchial tissues significantly increase the risk of relapse (p=0.035 and p<0.001, respectively). Multivariate analysis and a Kaplan-Meier curve showed that SP-A deletions in adjacent normal-appearing bronchial tissues is the most significant prognostic indicator for disease-specific survival (FIG. 7).

5). SP-A deletions may serve as useful markers for identifying patients with a poor prognosis and selecting patients with early-stage NSCLC who might benefit from adjuvant treatment.

All above discoveries provide a strong rationale for using these genetic signatures as biomarkers and represent advances in early lung cancer detection.

### Example 2

Generation of Specific Genomic Probes for the Candidate Genomic Signatures

Most DNA probes, including commercially available ones, are made from large genomic fragments cloned in a variety of vectors, including P1-derived artificial chromosome (PAC), BAC, and yeast artificial chromosome (YAC). The probes can target subchromosomal and band specific genomic regions ranging in length from 80-100 kb (PAC) or 100 to 200 kb (BAC) to 1 to 2 mb (YAC), thus producing clear, bright signals. However, these subchromosomal or chromosomal band-specific DNA probes are too large to detect single-gene deletions and amplifications in morphologic normal-appearing epithelium or hyperplasia in the early stages of lung carcinogenesis.

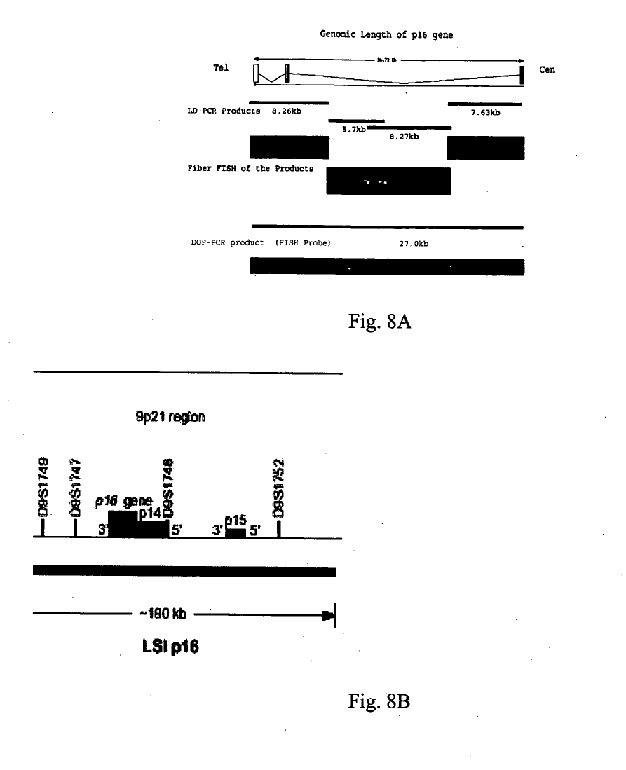

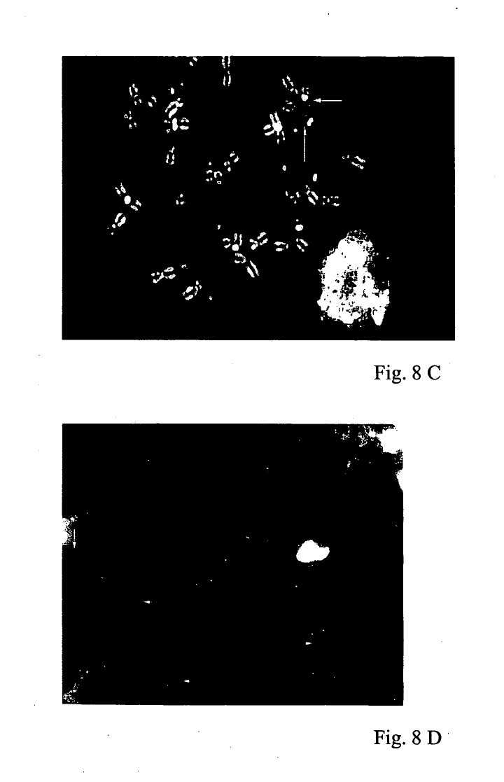

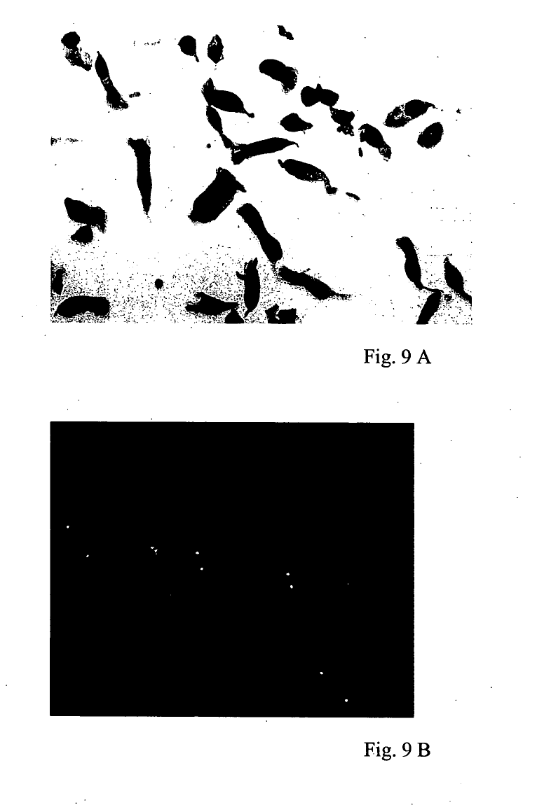

Development of specific genetic probes that exactly cover the full-length genome of a target gene is mandatory to overcome this problem (26). Recently, we developed a novel technique to accomplish this by performing a complex protocol. In the first step, considerably larger (up to 11 kb) amplimeres are amplified by long-distance polymerase chain reaction (LD-PCR) with genomic DNA extracted from the known BAC clone containing the gene of interest. With this approach, several overlapping bidirectional PCRs will structurally complex with genomic sequences that exactly span the whole genomic region of the target gene. Second, DNA of the LD-PCR amplicons is amplified by degenerate oligonucleotide primed-PCR (DOP-PCR), which is an efficient, reliable whole genome amplification technique that produces a large amount of highly reproducible specific genomic DNA for the target gene. Finally, the DOP-PCR-amplified DNA is labeled by a nick translation reaction with any available fluorochrome-dUTP. Using this novel approach, we first developed a 27-kb specific gene probe covering the full-length genomic p16 gene, which is located on chromosome 9p21.3, spanning a 26.72-kb genomic sequence (FIG. 8). We analyzed deletions of p16 on 11 NSCLC cell lines with the probe and with LS1p16 (Vysis, Downers Grove, Ill.), a commercially available p16 FISH probe that is 190 kb long and contains not only p16 (INK4A) but also several genetic loci, including D9S1749, D9S1747,p14 (ARF), D9S1748, p1 5 (INK4B), and D9S 1752. Compared with the LSI p16 probe, our unique p16 probe was more sensitive (66% versus 38%) in detecting the p16 deletion. The sensitivity was confirmed by Southern blotting with specific cDNA probes in the same sample. Thus, the specific gene probes can overcome the low detection efficiency of the conventional DNA probes.

Using this novel technique, we have generated specific genomic probes for all 40 candidate genomic signatures listed in Table 1. The probes have been tested in a variety of specimens and shown to have bright signals and right chromosomal location (FIG. 8).

Prospective detection of tumor cells with M-FISH in bronchoalveolar lavage (BAL) fluid samples obtained from patients at high risk for lung cancer

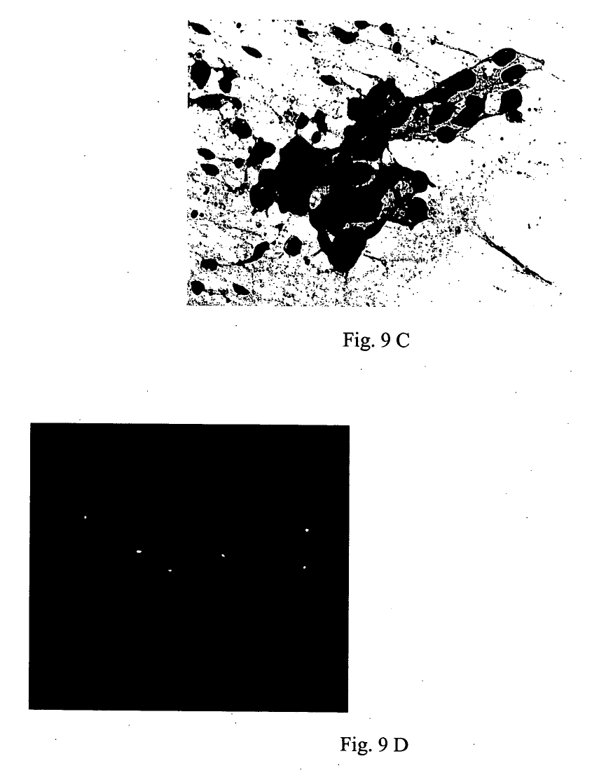

We analyzed numeric abnormalities of chromosomes 3 (red), 7 (green), and 17 (aqua) and aberrations of 9p (gold) in BAL samples prospectively obtained from 20 former smokers and 11 never smokers using the four-color M-FISH assay (LAVysion, Vysis). We found significant differences in the frequency of genomic instabilities among never, light, and heavy smokers. In BAL samples obtained from patients with lung cancer, the number of cells with the genomic aberrations correlated with the cytologic grade and the presence of malignancy (FIG. 9). BAL samples obtained from seven smokers subsequently diagnosed with lung malignancy showed genomic abnormalities, whereas only two showed morphologically atypical bronchial cells upon cytologic examination.

Although the number of cases in this study was limited, our results indicated that genomic alterations occur in the airway of smokers and that such alterations are related to an increased risk of lung cancer. Our results strongly support the previous findings showing that unique genetic alterations underlying the progression of lung carcinogenesis may be useful in the early detection of lung cancer. In addition, M-FISH may be more sensitive than conventional cytologic diagnosis in detecting deletions of the molecular abnormalities in BAL fluids.

2. M-FISH with tumor-specific gene probes has higher sensitivity and specificity in the early detection of lung cancer than does M-FISH with chromosomal or subchromosomal genomic probes

We performed a FISH study by using specific full-length DNA probes for GC20 (3p21.3) and SFTPA1 (10q22) and a commercially available four-color DNA probe set composed of 5q, 7p, 8q and 6 centromeres (LAVysion) to detect lung cancer cells and identify patients with early preneoplastic bronchial epithelial cell lesions at high risk for lung carcinoma. We prospectively analyzed bronchial brushings obtained from an area ipsilateral to the tumor, tumor touch imprints, and adjacent bronchial touch imprints of subsequently resected early-stage NSCLC from 30 patients. Bronchial washing specimens from patients without lung tumors were used as controls. Cytologic evaluation of the bronchial brushings showed basal cell hyperplasia, squamous metaplasia, or dysplasia in 28 of the 30 cases. FISH analysis showed that none of the bronchial brushings were positive for malignant cells that brushings were positive for up to 5 abnormal cells in 10 cases with LAVysion; however, 26 of the 30 cases showed deletions of both GC20 and SFTPA1. Among these patients, the incidence of positivity was 57% with LAVysion, 100% for GC20, and 79% for SFTPA1. LAVysion showed that the GC20 deletion level was 3.7 times higher, the SFTPA1 deletion level was 2 times higher, and the incidence of abnormal cells was 12.6 times higher in the tumor cells than in the bronchial epithelium on the bronchial brushings (28). In this study, the sensitivity in detecting the specific genes GC20 and SFTPA1 was at least two times higher than that of the chromosomal and chromosomal band-specific DNA probes in the bronchial brushings (Table 2.). Again, the results demonstrate that tumor-specific gene probes are more sensitive and specific in the early detection of lung cancer than are chromosomal and subchromosomal genomic probes.

TABLE 2
Logistic regression models were used to estimate and predict
patients' cancer status according to the probes' levels in tumor (T)
and tumor-side bronchial brushings (B) obtained
from 30 patients with lung cancer.

| | Parameter | Standard | | Odds |
|---|---|---|---|---|
| Probe/tissue | estimate | error | P value | ratio |
| GC20/B | 0.84 | 0.35 | 0.018 | 2.32 |
| GC20/T | 4.07 | 2.22 | <0.0001 | 58.3 |
| SP-A/B | 1.42 | 0.56 | 0.01 | 4.13 |
| SP-A/T | 1.42 | 0.56 | 0.01 | 4.13 |
| LAVy/T | 0.73 | 0.50 | 0.15 | 2.08 |

LAVy, LAVysion.

3. Automated Fluorescence Microscopy

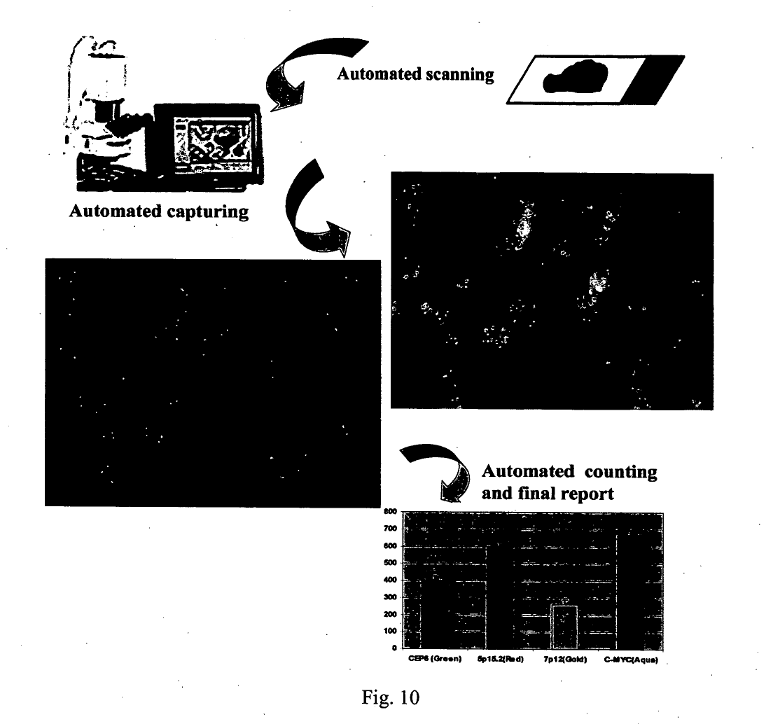

Dot counting is considered one of the most important applications of in situ hybridization, because the number of dots on a given image represents the number of inspected gene copies. Automatic dot-counting techniques are helpful in the analysis of large number of slides and cells, as manual counting is tedious and time consuming. We have used a commercial automated dot counting system, the CytoVision automated workstation (Applied Imaging), to exam bronchial brushings obtained from patients with lung cancer. We have also tested the LAVysion (Vysis), which consisted of one centromeric probe of chromosome 6 labeled in Aqua and three locus-specific probes for 5p15.2, 7p12, and 8q24.12 labeled with green, red, and gold, respectively. The results suggested that this unique system can greatly reduce the dot-counting time from 120 minutes with manual counting to 10 minutes per slide in counting 200 cells in the same specimens, with an accuracy similar to that of the manual method (FIG. 10). This automated dot-counting system contains all of the components common to image processing and analysis: automated focusing, image acquisition, segmentation, measurement, and classification. It may generate case reports with preferred data layouts, present results such as bar charts, and add laboratory logos to final reports, which may allow early diagnosis of lung cancer when using a single chip and provide accurate results within a few hours. All of these features will be greatly beneficial in the application of our in situ genomic DNA chip for simultaneous detection of multiple genetic aberrations.

Our experience in using this imaging equipment to measure biomarkers in clinical specimens has been demonstrated (46-51). In addition, to fully use the automated workstation in incorporating multiple fluorescence probes capable of determining the genomic signature in clinical specimens, we recently developed an adjustable-threshold algorithm to efficiently segment fluorescently labeled objects contained within three-dimensional (3D) images obtained under laser scanning confocal or two-photon microscope. This algorithm is accurate in identifying, counting and determining the location of FISH signals in the images (52).

The existing interdepartmental lung bank samples, data files, and IRB approved protocol can be used for performing the present invention.

1). Lung Sample Bank 1

A retrospective bank of fresh and paraffin embedded blocks, this portion of our interdepartmental bank contains 160 samples collected retrospectively from 1987 to 1989 frozen lung tumors and matching paraffin blocks. The majority of the patients had 5-8 years of follow-up data. Most importantly, the lung TMAs had been constructed from the blocks containing tumors, adjacent histologic normal-appearing bronchial epithelium, and normal bronchial epithelium obtained from 120 patients with stage I lung cancer.

2). Lung Sample Bank 2

This portion of the bank contains a prospective case series and obtain biologic samples and corresponding epidemiologic and clinical data. Over the past 3 years, under our IRB approved protocol for the creation of an aerodigestive tissue resource (LAB02-597, updated with the number FWA363), we have recruited 120 patients with newly diagnosed, previously untreated NSCLC who were scheduled to undergo surgery. Oral, sputum, and bronchial brush brushings, and tumor tissues were obtained. Additionally, oral brushing, sputum, and bronchial brush specimens were collected from 35 individuals with no history of smoking or lung cancer who were recruited from a chemoprevention trial at our institution. Cytospin preparations were made from the brushing via cytocentrifugation, and touch preparations were made from tumor tissues. All of the slides were immediately fixed in Carnoy's solution for 20 minutes and then stored in freezer at −80° C. All patients had stage I cancer. About 10% of our general patient population is Hispanic, and 10% is African American. A variety of demographic and clinical information about these patients, including but not limited to age, ethnicity, sex, tumor stage, and smoking status, has been collected.

### Example 3

Identification of a Set of Specific Genetic Probes that Provide the Greatest Overall Sensitivity and Specificity for Detection of Lung Cancer Cells

We have identified genetic signatures that correlate directly with early neoplastic progression of lung cancer and developed specific genetic probes for these signatures. The genetic signatures constitute biomarkers in early lung cancer diagnosis, and the specific genetic probes will be evaluated in normal, preinvasive, and early invasive neoplastic lung tissues.

We will test these specific probes with lung TMAs containing archived tumors, adjacent normal-appearing bronchial epithelium, and normal bronchial epithelium obtained from 120 patients with stage I lung cancer to develop a set of probes that provide the greatest overall sensitivity in lung cancer cell detection using the smallest number of probes.

Methods and Procedures:

Dual-color FISH: Corresponding chromosomal centromeric probes (CEP 1-22 and X) labeled with green-dUTP fluorochrome will be used as internal controls for each specific genetic probe. All the specific probes will be labeled with red-dUTP fluorochrome as described in a previous study by our group (56). A total of 40 candidate probes will be tested as shown in Table 1. Dual-color FISH tests of the TMAs will be performed as described in our previous studies (26, 42, 56-59). Briefly, an LSI hybridization buffer (Vysis) will be used according to the manufacturer's instructions. Ten microliters of probe mixture will be added to each slide. HYBrite (Vysis) will be used to denature the cells at 74° C. for 4.5 minutes and hybridize them at 37° C. overnight. Post hybridization washing will be performed with 50% formamide plus 2× standard sodium citrate (SSC) for 10 minutes at 43° C. three times followed by 2×SSC for 10 min at 43° C. and 2×SSC plus 0.1% NP-40 for 5 min at 43° C. Finally, the cells will be counterstained with 0.2 μM 4, 6-diamidino-2-phenylindole ( DAPI). Enumeration of FISH signals will be performed under a Leica fluorescence microscope (Leica, Deerfield, Ill.) equipped with a filter set (green, red, and blue) and cooled CCD camera (Photometrics, Tucson, Ariz.)

Scoring criteria: The criteria for enumerating of FISH signals were described in our previous publications (26, 42, 56-59). The criteria for gene amplification are tight clusters of signals in multiple cells or at least 2.5 times more test probe signals than CEP signals per cell in more than 10% of the tumor cells. The criterion for gene deletion is a ratio of test probe/CEP signals less than 1. The cutoff value will be calculated with normal tissue samples using the mean number of cells having an abnormal FISH signal pattern plus three standard deviations.

Statistical analysis: Correlations between the ratio of test probe/CEP signals and the patients' clinical and demographic features will be explored. Categorical variables will be summarized with the use of contingency tables. Odds ratios and their corresponding 95% confidence intervals will be reported for categorical variables. Kaplan-Meier curves and the Cox proportional hazards model will be used to analyze survival data. As a continuous variable, Student's t-test will be used to assess differences in the average number of aberrations between groups defined by demographic or clinical characteristics. Complementation analysis will be performed to make definitive conclusions about the clinic-pathologic relevance of each of the 40 probes and identify a set of probes that provide the greatest overall sensitivity with the smallest number of probes.

Gene aberrations can occur in both normal-appearing bronchial epithelium and, to a much higher degree, carcinoma cells. Aberrations represented by each probe may exist in 30%-40% of the tumor cells. If the aberrations detected by individual markers are independent, based on complementation analysis, a combination of six to eight selected probes will be required to achieve sensitivity and specificity of close to 100% in the detection of cancer cells.

FISH tests with paraffin-embedded sections of TMAs may have some artifacts as a result of truncated nuclei, thick section preparations, and autofluroescence of some tissues, which may obscure the detection of signals. We will reduce the potential for artifacts in M-FISH by using our Open Lab imaging system with a confocal function, which is used for 3D analysis and provides complete, accurate registration of fluorescent signals on cells by simultaneously scanning through separate filter blocks. Such small probes may produce weak signals, especially in some poor-quality paraffin embedded tissues. In such case, we will use peroxidase-driven tyramide signal amplification to amplify the probes, which will greatly reduce the non specific background and increase the intensity of the signals (60).

### Example 4

Development of an In Situ Genomic DNA Array Consisting of the Most Clinicopathologically Relevant Probes.

A conventional M-FISH assay using chromosomal probes to detect cancer cells in bronchial washings is superior to that of conventional cytologic methods in the detection of cancer cells (27). Therefore, the in situ DNA array, using a relevant panel of specific probes for the genes underlying lung tumorigenesis, may be a much more practical molecular test in a clinical laboratory setting and more clinically applicable assay for early detection of lung malignancy. Furthermore, because lung cancer is a heterogeneous disease featuring field defects in the airway of smokers, a single biomarker is insufficient to detect lung cancer with acceptable accuracy (1). The in situ genomic DNA array, when used with multiple probes for the detection of molecular alterations unique to lung carcinogenesis, will be much more accurate.

Methods and Procedures:

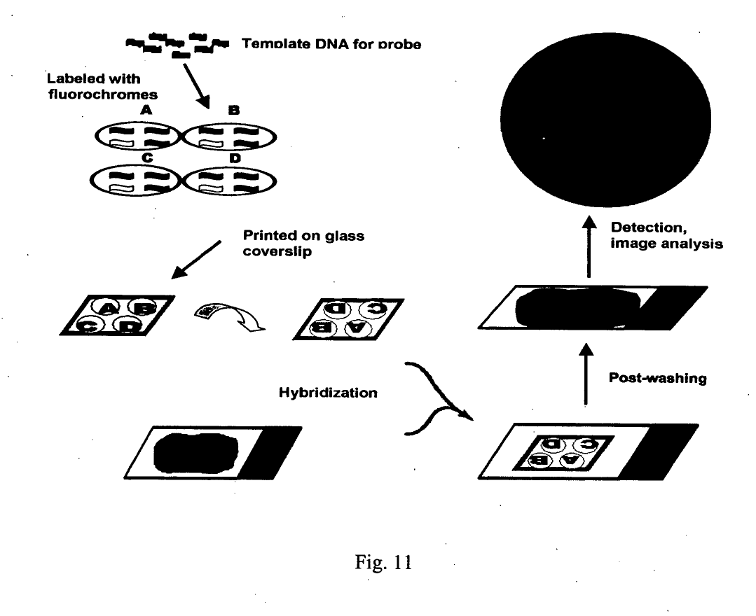

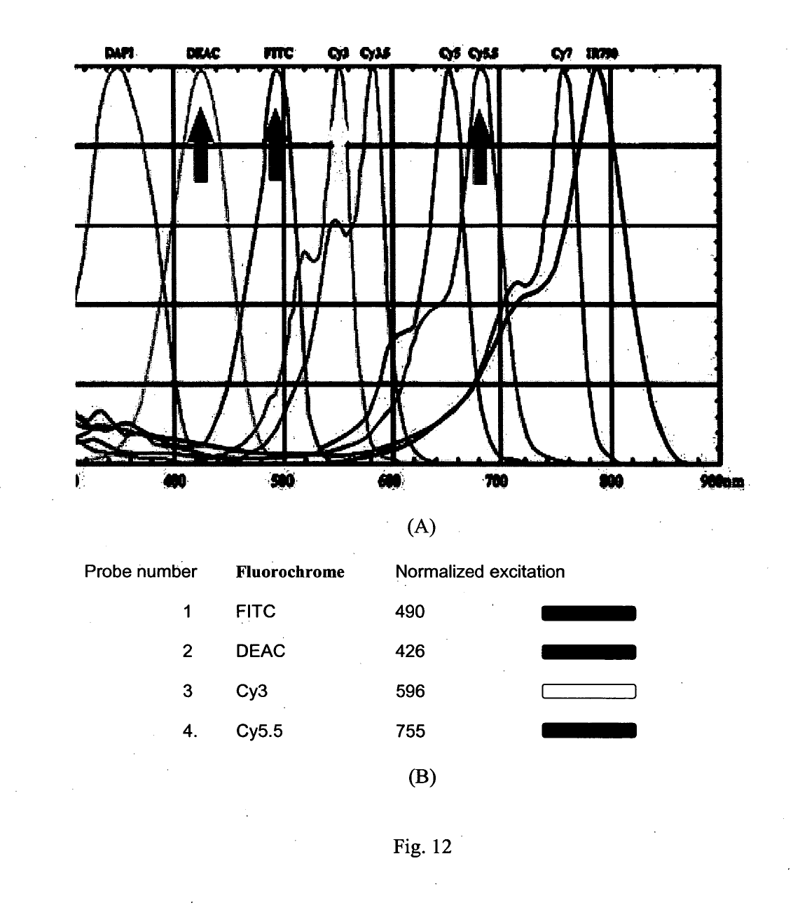

1. Designing and Manufacturing the In Situ Genomic DNA Chip (FIG. 11-12):

Labeling probes and making the array. Template DNAs for genes of interest are labeled with four fluorochromes by nick translation. The probes are arranged in four elements. Each element contains a cocktail of four probes that will be ethanol-precipitated and printed on a coated glass coverslip.

Hybridization. After the hybridization solution is added to the slide, the coverslip is inverted over the slide and sealed with rubber cement. Four corresponding cycles will be marked on the back of the slide according to the location of the four elements on the coverslip with a diamond pen. Following denaturation, hybridization will be performed overnight with the Hybrite hybridization system.

Postwashing and detection. The slide is washed in 50% formamide. Images will be captured with microscopes equipped with a set of eight fluorescent filters based on the cycles on the slide, and FISH signals will be counted with a CCD camera and pseudo colored with the OpenLab imaging software program.

The number of probes corresponding to each gene used in a cocktail of the DNA chip will depend on the complementation analysis in Specific Aim 1, which will determine how many probes will be used in combination to provide the highest sensitivity. We predict that a combination of six to eight probes in a single assay will result in a sensitivity and specificity of 100%. Therefore, together with their corresponding chromosomal centromeric probes, which will be used as internal controls; there will be 12-16 probes on the chip. Four different compositions of cocktails (elements) of four probes for each are bound on the same coverslip as shown in FIG. 11, each element will contain four probes, which will be labeled in red, green, aqua, and gold, respectively. Because there is no overlap of wavelengths among the four fluorochromes (FIG. 12), the signals will be distinguished clearly from each other under appropriate filters.

A cocktail of probes will be ethanol-precipitated, resuspended in a matrix of glass-immobilized gel elements by polymerization of 20-mm-thin polyacrylamide gel (8% acrylamidey 0.28% bisacrylamide), and reversibly bound to a conventional 36×36-mm glass coverslip (chip) (FIG. 12). The chip will then be ready for hybridization. The unique methodologies and wide space between each element array design will prevent mixing of the probes from different elements on the array during hybridization.

2. Determining the Performance of the In Situ Genomic DNA-Chip

Cells from lung cancer cell lines will be mixed with normal epithelial cells to produce various serial cell dilutions. Mononuclear cells will be fixed in Carnoy's solution. A slide will be carefully inverted over the in situ genomic chip, which will be positioned upside down (FIG. 12). Hybridization and postwashing will be performed under the same protocol as described in our published work (56). Images will be captured under Leica fluorescent microscopes.

3. Defining the Cutoff Value for a Positive Result and Data Analysis plan

A cell will be defined as abnormal if it has three or more copies of any probe signal or if there is heterozygous or homozygous loss of any signal as compared with its internal controls.

4. Results

Multiple genetic aberrations will be identified because the set of four fluorescent filters will allow a high degree of discrimination between the four fluorochrome-labeled probes. The normal limit for each probe will be set according to the mean percentage and standard deviation of abnormal cells among the normal control cells, which will differ significantly from those among tumor cells. Some techniques including manufacturing DNA arrays on glass, have been advanced at both our primary and collaborative laboratory (53-55). The test using the slides prepared from fresh cell lines will be very easy to perform and will always produce bright, distinct signals.

### Example 5

Characterization of the Performance of the In Situ Genomic DNA Array with Easily Accessible Tissues by Analyzing Surrogate and Final Target Tissues in the Respiratory Tract for Genetic Abnormalities and Correlating these Data with Clinical Covariates, Especially the Results of Cytologic Studies

A major obstacle in conducting studies of means of detecting early-stage lung cancer is obtaining appropriate cellular tissue specimens. Sputum and buccal brushings are easily and inexpensively obtained. On the basis of field cancerization, concordance of the degree of genetic aberrations in all of the target tissues, such as in respiratory epithelial cells exfoliated from sputum and oral brushings and lung cancer tissue, is contemplated. The in situ technique will work particularly well with sputum and buccal brushings because such samples are limited in cellularity and their cells are arranged in monolayers. Accordingly, sputum and buccal brushing may replace bronchial biopsy and lavage specimens and may be suitable for assessing carcinogenic damage in lung cancer as surrogate tissue.

Test for genetic changes in oral, sputum, and bronchial brushings and ultimate target lung tissues using the in situ genomic array. The concordance of the findings are assessed to determine the sensitivity and specificity of the assay and identify its diagnostic criteria in the oral and sputum specimens. The genetic aberrations detected in the surrogate tissues by the assay are compared with the results of multiple clinic-pathogenetic criteria, especially cytologic diagnosis, to evaluate the clinical significance of the assay.

Methods and Procedures:

1. Cytopathologic evaluation: Papanicolaou's preparations from sputum, buccal, and bronchial brushings and tumors will be obtained from our lung tissue bank and evaluated by two of the co-PIs, who are experienced pathologists for cytologic criteria and histopathologic findings for the primary tumor.

2. Genetic aberration analysis with in situ genomic DNA array: Genetic aberrations will be detected with the genomic DNA array in specimens obtained from a prospective case series of 120 patients with lung cancer and 35 individuals without lung cancer (Table 3). Experiments will be performed under the protocol described for Specific Aim 2. The same criteria and cutoff value established in Specific Aim 2 will be used for signal analysis.

TABLE 3
Experimental design for Specific Aim 3 and 4

| | | | |
|---|---|---|---|
| | Buccal | Bronchial | |
| Sputum^{a} | brushings^{a} | brushings^{a} | Tumor^{b} |

Specimens obtained from 120 patients with lung cancer

| | | | |
|---|---|---|---|
| 120^{c} | 120^{c} | 120^{c} | 120^{c} |

Specimens from obtained from 35 individuals without lung cancer.

| | | | |
|---|---|---|---|
| 35^{c} | 35^{c} | 35^{c} | 0 |

^{a}Cytospins,
^{b}Touch preparations,
^{c}The number of specimens will be tested with the in situ genomic DNA array.

3. Statistical Analysis:

For array analysis of oral, sputum and bronchial brushings and tumor tissue specimens, we will record the number of aberrations per 200 interphases per subject using the cutoff value, which will be calculated in normal respiratory epithelial cells.

Pearson's correlation coefficient will be used to measure the correlation between the tumor and surrogate tissue aberration levels of each gene. Using a general linear model, we will then explore the relationship between the number of genetic aberrations in tumor tissue and that in the cells obtained from the oral, sputum, and bronchial brushings. For power calculations, we will assume that the null hypothesis is that there is a modest correlation (ρ₀=0.5) between the number of aberrations in these two types of tissue. A 0.05 two-sided Fisher's test of the null hypothesis with a Pearson's correlation coefficient ρ₀=0.5 shows that a sample size of 120 will have 85% power to detect a ρ of 0.5. We will apply chi-square cross-tabulation tests to examine associations between aberrations detected by the in situ genomic DNA array in the cells obtained from the oral and sputum brushings. We will perform the Cox proportional hazards model for this panel of markers in the assay and compare the results for multiple clinicopathogenetic criteria, including those of a cytologic study, to evaluate the clinical significance of the assay and diagnostic criteria in surrogate tissues.

4. Methodology: An association is contemplated between genetic aberrations in lung cancer tissues and those in sputum and buccal epithelial, bronchical epithelial, and target lung cancer tissues. The percentage of cells with abnormal signals are contemplated to correlate with important clinicopathologic features. Correlation of the genetic aberrations found in these tissues may vary from absolute accuracy because, for example, site variations can create a certain degree of inconsistency owing to the nature of small, independently occurring abnormal clones in the defective fields. For example, single genetic aberrations occur in bronchial epithelium and, to a much higher degree, carcinoma tissues, whereas the overall genetic changes in bronchial epithelium are similar to those in tumors obtained from the same patients. However, the sensitivity and specificity of the assay in the detection of lung cancer cells is around 80, 81, 82, 83, 84, 85, 86, 87, 88, 89, 90, 91, 92, 93, 94, 95, 96, 97, 98, or 99%.

### Example 6

Development of an In Situ DNA Genomic Array as an Automated Genetic Test Using Automation of Fluorescent Dot Counting

The in situ genomic DNA array assay will be combined with an automated dot counting system, and the genetic signatures will be tested with oral and sputum brushings to develop the assay as an automated genetic test. All of the hybridized slides of buccal and sputum smears (Table 3) described in Specific Aim 3 that have been analyzed manually will be counted using the CytoVision SPOT AX automated workstation. The slides will be screened with the system according to the manufacturer's instructions (FIG. 10). After analyzing the required 200 cells per slide, we will intervene to correct the computer by examining a gallery of the cell images. The performance of the assay will also be tested with normal specimens showing normal distribution of each signal (two dots for each signal). The signal screening and capturing procedure will be performed with CytoVision workstation as described above.

2. Scoring criteria and definition of the cutoff value for a positive FISH result: The scoring criteria and definition of the cutoff value for a positive FISH result will be the same as those used for manual counting in Specific Aim 2.

3. Statistical analysis: The McNemar test for correlated proportions will be used to determine the difference in sensitivity between automated and manual dot counting. The p value for the difference in specificity between the two methods will be determined using the generalized estimating equations technique. The Cox proportional hazards model will be performed for this panel of markers in the assay, the results of which will be compared with those of the cytologic study to evaluate the clinical significance of the assay and the diagnostic criteria in surrogate tissues.

### Example 7

Development of In Situ Genomic DNA Array Capable of Detecting a Panel of Genetic Aberrations with Multiple Specific Probes

Based on our design of array techniques and taking advantage of in situ hybridization and multicolor labeling, we developed an in situ genomic DNA array that can not only be used directly on clinical specimens, but can also simultaneously detect a panel of unique genetic aberrations with multiple specific probes. We first generated gene-specific genomic probes for the most clinicpathological relevant genetic signatures in primary lung cancer and designed an in situ DNA probe chip consisting of the probes. We then tested the chip on bronchial brushings from lung cancer patients to detect genetic abnormalities and compared the data with results of cytologic studies and of a commercial four-color FISH set probes.

MATERIALS AND METHODS

1. Patient and Control Population and Specimen Preparation

Forty patients with stage I non-small lung cancer were entered into our prospective study under an institutional review board-approved protocol to create an aerodigestive tissue resource. All patients had a lung mass that was operable. The patients underwent bronchoscopy just before surgical excision. Bronchial brushings were taken from the mainstem bronchus on the tumor-bearing side. Normal lymphocytes from six healthy individuals and bronchial washings from 20 individuals with no history of smoking or lung cancer were used as controls. Cell suspensions of bronchial washings were dropped onto the slides and fixed in Carnoy's solution (methanol to acetic acid, 3:1) for 20 min and then stored in freezer at −80° C., as previously described.

2. Designing a Cancer-Specific Genomic Probe Panel

A panel of eight specific genomic probes and their corresponding chromosomal centromeric probes were used to design the in situ DNA chip (Table 4). The genes were selected on the basis of primary lung cancer-specific genomic signatures, which were detected by a microarray-based comparative genomic hybridization analysis. To develop the unique genomic probes that would specifically cover the genomic sequences of the selected genes, two strategies were used. First, for the genes with a large genomic length that potentially showed increased copy number changes in lung cancer cells, several gene databases, including the Celera database (http://www.celera.com) and the Ensembl human genome database (http://www.ensembl.org) were searched to identify BAC or PAC clones containing only the genomic sequences of the genes of interest. After obtaining the relevant clones (Invitrogen Corporation, Carlsbad, Calif.), inset DNA was prepared to use as a probe, as previously described.

Second, to develop a specific genomic probe that exactly covered the full length of the genome for the genes with a small genomic size, especially those potentially deleted in cancer cells, clones that might contain genomic sequences of several genes were identified including the specific gene of interest. Considerably larger (1-10 kb) amplimers were then amplified by long-distance PCR (LD-PCR) with specific primers for the target gene from the clone DNA. With this approach, several overlapping bidirectional PCRs with structurally complex genomic sequences were produced to span the whole genomic region of the target gene. The DNA of the LD-PCR amplicons was then amplified by degenerate oligonucleotide-primed PCR (DOP-PCR) (DOP PCR Master; Roche Diagnostics Corporation, Indianapolis, Ind.), according to the manufacturer's instruction; this was an efficient, reliable whole-genome amplification technique that produced a large amount of highly reproducible, specific genomic DNA for the target gene. Finally, the DOP-PCR-amplified DNA was labeled by a nick translation reaction with fluorochrome-dUTP (Alexa Fluor; Molecular Probes, Eugene, Oreg.) (Table 4), as previously described. The hybridization efficiency of each probe was tested and validated on the chromosomal spreads of normal lymphocytes and their corresponding chromosomal centromeric probes (Vysis, Inc., and Qbiogene, Irvine, Calif.), which were used as controls. To further validate the size and the mapping region of the gene-specific genomic probes developed by the LD-PCR and DOP-PCR protocols, segments of the DNA products and final probe were analyzed on normal DNA fiber preparations, as previously described.

3. Designing and Manufacturing the in Situ Genomic DNA Chip

The DNA chip consisted of 16 probes, including the gene-specific probes and their corresponding chromosomal centromeric probes. Four different compositions of cocktails of four probes each were arrayed (FIG. 11 and Table 4). These four probes were labeled in red, green, aqua, and yellow (Table 4). All the chromosomal centromeric probes were commercially available (Vysis, Inc., and Qbiogene). One side of a conventional 36×36-mm glass coverslip was divided into four separate squares of equal size by mounting on a high-melt agarose gel (FIG. 11); each square was 8×8 mm and marked as square A, B, C, or D on the back side of the coverslip to indicate its location. The distance between each square was 6 mm. Four different cocktails of probes were precipitated with ethanol. Each cocktail contained 100 ng of each probe mixed with a 30-fold excess of human Cot-1 DNA (Life Technologies, Rockville, Md.) and was resuspended after precipitation in 6 μl of a matrix of glass-immobilized gel elements by polymerization of a 20-mm-thin 8% polyacrylamide gel (Sigma-Aldrich, Saint Louis, Mo.); this polymerization reversibly bound each square on the glass coverslip (FIG. 11). After 10 min, the matrix of glass-immobilized gel elements containing the probes became solid at room temperature, while the gel remained soluble and allowed the probes to hybridize on the target cells at a temperature of 37° C. or above. However, the separate high-melt agarose gel did not melt at 100° C., which prevented the mixing of probes from different squares during denaturation and hybridization.

Slides with mononuclear cells were mounted in a 20-μl hybridization buffer (Vysis) and then were covered by inverting over the in situ genomic chip, which was positioned upside down (FIG. 11). After the edge of the coverslip was sealed with rubber cement (Sigma-Aldrich), the location of each square was also marked on the back side of the slide with a diamond pen, corresponding to the previously marked location on the back side of the coverslip. The hybridization was performed on the HYBrite™ Denaturation/Hybridization unit (Vysis) according to the manufacturer's instructions with the following program: denaturing at 74° C. for 4.5 min and hybridization at 37° C. overnight. Post-washing was performed under the conventional FISH protocol described in our previously published study. Finally, the cells were counterstained with 0.2 μM of 4′, 6-diamidino-2-phenylindole (DAPI). In addition, to test whether the separation methodologies used and the space between each element of the array design would prevent the mixing of probes from different elements on the chip during hybridization, the centromeric probes for chromosomes 1 (in red; Qbiogene), 2 (in green; Qbiogene), 3 (in aqua; Vysis), and 4 (in orange; Vysis) were added to each square of the coverslip. These probes were then hybridized on five normal control and five cancer specimens.

To evaluate the performance of the chip, a commercial LAVysion four-color probe set (Vysis) that targeted centromere 6, 5p15.2, 7p12 (EGFR), and 8q24 (MYC) was tested on the same specimens in parallel using the same protocol described in our previous study. For cytologic diagnosis, Papanicolaou-stained and Diff-Quik-stained preparations were also made from each bronchial washing specimen in parallel with the slides fixed in Carnoy's solution. Papanicolaou preparations and Diff-Quik slides were then reviewed independently by two pathologists (R.L.K and N.P.C).

4. Epifluorescence Microscopy

Images from the chip and LAVysion probe set assays were obtained with motorized epifluorescence microscopy (Leica Microsystems Inc, Bannockburn, Ill.). The microscope was equipped with a filter set (Chroma Technology Corp., Brattleboro, Vt.) that included DAPI, aqua, yellow, red, and green single bandpasses, which were specific for the four spectrally distinct fluorochromes used for the probes in the chip and LAVysion probe set assay. To count the signals from the LAVysion probe set, at least 100 cells were analyzed, as previously described. The signals from the chip were counted in a similar manner, and the number of aberrations per 100 interphases per square was recorded from square A through square D serially, according to the marked line on the back side of the slides. Both the camera and the microscope were controlled by software (Openlab; Scientific Software, Inc., Pleasanton, Calif.).

Two criteria were used for considering a cell abnormal: 1) the cell had to have three or more copies or exhibit hemizygous or homozygous loss of a specific gene probe signal, compared with its corresponding chromosomal centromeric probe; and 2) although having no loss of any specific gene probe, as described in the first criterion, the cell had to have an aneusomy (such as monosomy, trisomy, or tertrasomy) of any centromeric probe.

5. Statistical Analysis

The sensitivity and specificity of all assays were calculated by using receiver operating characteristic (ROC) analysis as previously described. To be conservative in estimating the percentage of abnormal specimens, the optimal cutoff chosen was the one with a 0% false-positive rate or, equivalently, 100% specificity. χ² tests were used to compare the results of the in situ genomic DNA chip assay with the data generated by the LAVysion multicolor probe set and cytologic assays.

Results

1. Generation and Validation of the Hybridization Efficiency of the Gene-Specific Genomic Probes

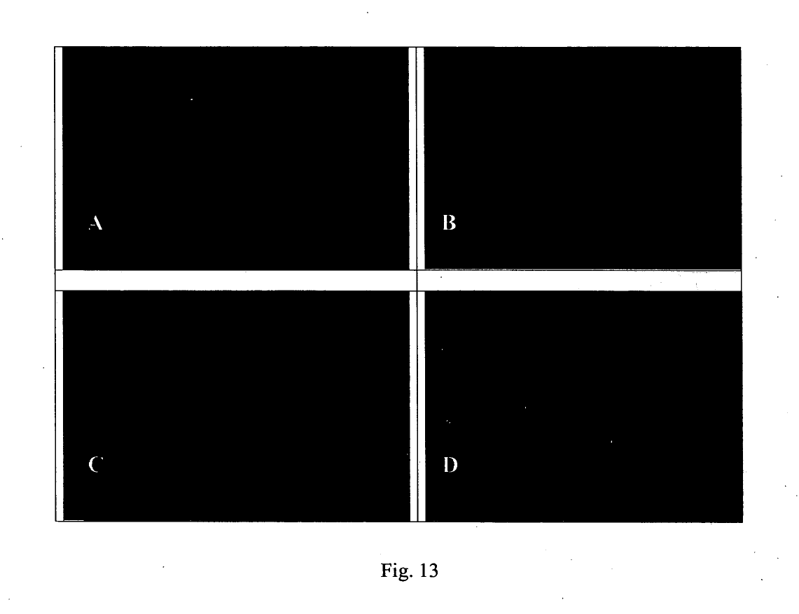

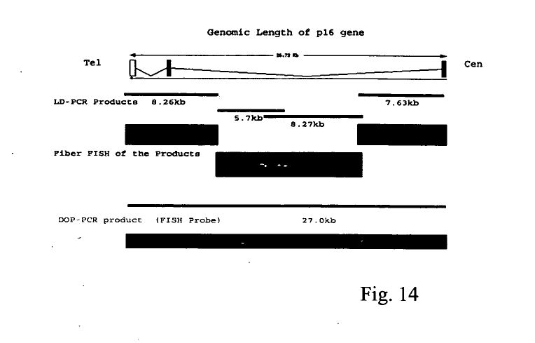

The precise location of each of the eight specific probes that covered the full length of the genomes of the eight genes of interest was confirmed by the normal metaphase chromosomal spreads of the genes and of the corresponding chromosomal centromeric probes. The specific signals of all the counted metaphases of each probe were labeled correctly, and the probes were hybridized in the correct chromosomal location. This was assessed by viewing the individual fluorochrome channels in a karyotype format, which indicated the absence of chimerical signals and the extreme brightness of the signals (FIG. 13). The hybridization efficiency (defined as the percentage of cells with fluorescent signals on both sister chromatids of both chromosomal homologs) of the probes on the normal metaphase spreads, was 100%, as was the hybridization efficiency of the commercial centromeric probes. There was no overlap of wavelengths among the four spectrally distinct fluorochromes: the signals were clearly distinguishable from each other under the appropriate filters. Furthermore, the exact size and mapping region of each gene-specific probe developed by the LD-DOP-PCR protocol was confirmed by FISH on normal DNA fiber, which showed a single linear DNA molecule with juxtaposed color barcode signals (FIG. 14).

2. Performance of the Chip and Definition of Its Cutoff Value

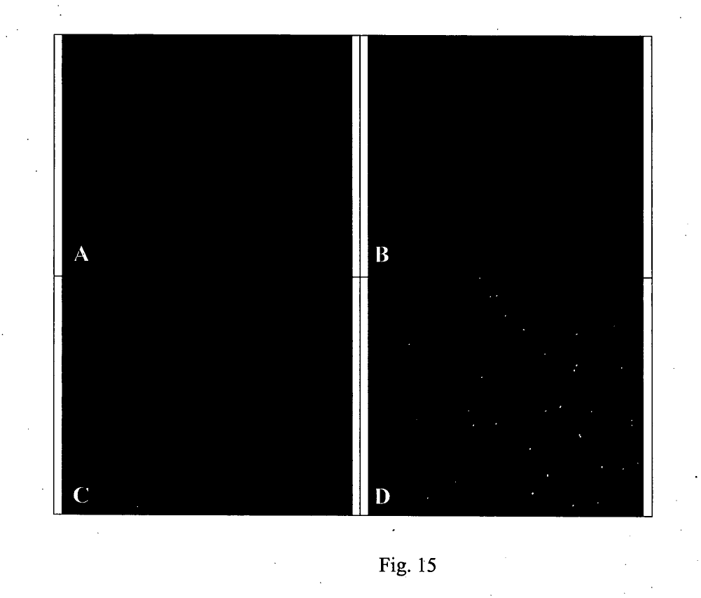

Normal lymphocytes from six healthy individuals displayed two signals (disomy) for each probe, as shown by the normal diploid population that comprised two aqua, two green, two red, and two yellow signals in each element detected. The chips containing only a one-color fluorescence-labeled probe in each element were also tested in 10 specimens. In all cases, square A showed only a red signal, square B a green signal, and so on (FIG. 15), indicating that no mixture of the probes occurred between different squares during hybridization.

To establish the cut-off value that determined whether a case was positive or negative for cancer, we calculated the number of abnormal cells in each specimen. An ROC curve was plotted for sensitivity as a function of 100% specificity, based on the percentage of abnormal cells. The optimal point in the assay corresponded to a cutoff value of six abnormal cells per specimen. Consequently, a specimen was classified as positive for cancer if it included six or more abnormal cells; otherwise the sample was considered negative for cancer.

3. Detection of Cancer Cells by the in situ DNA Chip and Comparison with Results of Conventional Cytologic and an IM-FISH Tests.

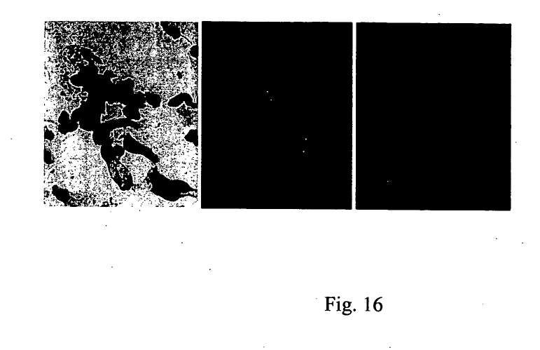

Of the 40 bronchial washing specimens from patients with a histologically confirmed diagnosis of lung cancer, 36 were classified as abnormal by the in situ DNA chip assay, indicating that this assay had a 90% sensitivity in detecting cancer cells. In the cancer-positive samples, the percentage of abnormal cells was variable, ranging from 11% to 78% (mean, 44.5%), which was statistically significantly higher than the 6% cutoff value (P=0.008). Using the same bronchial washing specimens, cytologic analysis suggested the presence of cancer in 21 (52.5%) cases and IM-FISH (LAVysion) in 26 (67.5%). Of the 19 cases of cancer not detected by cytologic analysis, the LAVysion kit and our in situ DNA chip assay detected 6 and 15 cases, respectively, suggesting that the use of molecular genetic diagnostic methods improved the accuracy of the diagnosis of cancer. Furthermore, in the 14 cases of LAVysion-negative specimens, 10 cases were positive according to the in situ DNA chip assay (FIG. 16), showing that the sensitivity of the chip assay for detecting cancer cells in bronchial washing specimens was statistically higher than that of either cytologic analysis or LAVysion (P=0.006 and P=0.003, respectively). None of the bronchial brushing specimens from the 20 individuals with no history of smoking or lung tumors were shown to have abnormal cells by cytologic analysis, the LAVysion kit, or the in situ DNA chip assay, suggesting that the specificity of the three tests was same (100% by design).

It is shown in the specimens tested that the sensitivity of the in situ DNA chip for simultaneously measuring a panel of biomarkers was markedly higher than the sensitivities of cytologic analysis and LAVysion IM-FISH, indicating that our assay represents a substantial improvement in the detection of lung carcinoma in bronchial washing specimens. Because the chip assay also had the same specificity as did cytologic analysis and LAVysion IM-FISH, it might be a powerful diagnostic test and a useful adjunct to cytologic analysis in the examination of bronchial secretions for evidence of cancer.

The sensitivity of a genetic assay is directly proportional to the number of probes used simultaneously. For example, the sensitivity of single-color FISH ranges between 10⁻¹ to 10⁻³, whereas the use of two to three DNA probes can increase this sensitivity to a range of 10⁻³ to 10⁻⁴. (Kasprzyk A et al., Leukemia. 11(3):429-35 (1997). As a result, IM-FISH has great promise for increasing the rate of cancer diagnosis at the single-cell level because it allows visualization of multiple probes simultaneously and, thus, streamlines the screening of specimens for chromosomal aneuploidies and/or copy number changes of specific cancer-associated genes.

There are currently two approaches for increasing the number of fluorophore-labeled probes used in IM-FISH and thereby overcoming the previously discussed limitations of available strategies. First, different fluorescence dyes can be used for either combinatorial labeling or ratio labeling. Although this approach can theoretically distinguish a greater number of targets for a given number of fluorescent labels, it is more complicated in terms of probe labeling, so more accurate fluorescence measurements are needed. For example, if hybridization intensities were poor or background and autofluorescence were high, especially in nuclei, a pair of ratios could be so close that the color difference between the targets would be too small to clearly distinguish between the targets, thus resulting in very high hybridization variability artifacts.

From the perspective of label, imaging, and filter set economy, the second approach is the least ambiguous and simplest method of multitarget analysis. In this technique, a different, spectrally distinct fluorescent label stains each probe in a single assay. Current epifluorescence microscopy consisting of as many as eight different color channels or filters can be used for the simultaneous analysis of multiple probes; however, not all eight targets could be visually distinguished with certainty based solely on their color when viewed directly through the microscope using any single bandpass filter sets; for example, probe stains range from green to orange, with the intermediate gradations of yellow and orange difficult to distinguish from one another. Even without these concerns, the size of the interphase also does not allow many probes to be hybridized simultaneously. For example, using a panel with as many as six probes in individual cells will result in very labor-intensive screening and great interpretative challenges when signals are counted in highly aneuploid samples; furthermore, focal plane distortions can occur because of either overlapping signals or the blending of fluorophores when the different signals were close to one another. Therefore, it is not worthwhile to simply increase the number of labeling probes arrayed in an interphase molecular cytogenetic diagnostic test.

In our study, rather than merely increasing the number of fluorochromes to achieve a reduction in probe complexity, we proposed instead a conceptual change by which a coverslip was divided in four small subregions, and four-color FISH experiments were performed in parallel. We clearly demonstrated that the unique separation methodologies used and the wide space left between each element of the array design prevented mixing of the probes from different elements during hybridization. Furthermore, to avoid any overlapping of adjacent fluorochromes due to significant overlap in the excitation and emission spectra, we chose green, red, aqua, and yellow fluorochromes in the array design. These four distinct dyes have the most discriminative wavelengths and have been shown to be the most stable and brightest fluorochromes; this prevented the possibility of overlapping, bleaching, or weakness of the hybridization signals. All genetic and chromosomal targets were classified correctly in metaphases, interphases, and DNA fiber.

Because the in situ DNA chip consists of fewer fluorochromes and requires less labeling, fewer filter sets, and fewer images to be recorded, both the image acquisition and image processing times are reduced. Furthermore, counting the signals of the in situ DNA chip requires only a conventional fluorescent microscope equipped with common fluorescent filters and a digital camera rather than more sophisticated imaging systems and software for performing analyses. This will greatly benefit clinical and research laboratories that lack access to the complicated and expensive equipment generally needed to perform molecular genetic studies. In particular, the strategy may satisfy the requirements of speed, simplicity, and reliability demanded of routine diagnostic tools in the clinical setting.

Most current DNA probes are made from large genomic fragments cloned from a variety of vectors, including PC (80-100 kb), BAC (100-200 kb), and YAC (1-2 mb), and only target subchromosomal and band-specific genomic regions. Therefore, these probes cannot detect cancer-specific genetic changes, especially single-gene deletions, which are, in most cases, specific genetic events that occur in preneoplastic cells or in the early stages of carcinogenesis. In contrast, we successfully generated specific genomic probes for the cancer-related genomic signatures. Furthermore, given that the size of some specific probes that have been developed are comparatively small, which can lead to false-negative results in the detection gene deletions because the signals of the genes may be artificially weak, Alexa fluorochrome were used to label the probes. These fluorochromes have been shown to possess superior brightness and photostability compared with conventional fluorochromes and have been successfully used to label cDNA probes. Even the smallest genomic probes (e.g., 2.3 kb for the FGF4 probe) labeled with Alexa fluorochromes in the test samples exhibited very bright signals with high hybridization sensitivity and efficiency compared with commercial centromeric probes in the detection of target genes. These results were also confirmed by the DNA fiber spreads, implying that the generated specific gene probes can overcome the low detection efficiency of conventional large-fragment DNA probes. Therefore, the genomic changes detected by these probes might better reflect the real genotypes of cancer cells.

For interphase molecular genetic diagnosis, careful selection of the probe type is another key to provide accurate diagnostic information. Quantitative DNA content measurement by a limited number of centromeric probes alone might not be sufficient in the diagnosis of cancer. However, detection of specific genetic changes can provide more sensitive results than can measurement of changes in the chromosomal copy number because the specific genetic changes usually occur earlier and are more specific signs of the initiation and progression of tumorigenesis than is chromosomal aneuploidy. For example, although the UroVysion kit has been shown to be more sensitive and specific than cytologic analysis in detecting the late stages of bladder cancer, it was not superior to cytologic analysis for the early detection of this cancer in urine or bladder washings because it consists mainly of centromeric probes and thus was unable to detect specific genetic aberrations. Furthermore, the simultaneous use of the corresponding centromeric probes mapped to the same chromosome on which the specific gene of interest is located is essential to detect specific genetic aberrations because without such a control, the specific genetic probe will have low sensitivity. For example, we previously used UroVysion to test urine samples and found that the probe for p16, which is located on chromosome 9p2, was surprisingly one of the least-sensitive probes, despite the fact that loss of p16 has been reported as the most frequent chromosomal abnormality in bladder cancer. An explanation of this paradoxical finding may be that the copy number of the p16 probe appears normal, even though the cell has a p16 deletion and tetraploidization of chromosome 9 or the cell is hyperdiploid. Because a chromosome 9 centromeric probe was not used as a control, the probes used might have given false-negative results regarding p16 deletion.

Similarly, although the LAVysion kit was designed to identify cells with changes of three selected chromosomal loci (5p15, 8q24, 7p12) and of the centromeric region of chromosome 1, which is used as an indicator of the ploidy status of the tested cell, no corresponding centromeric probe for chromosomes 5, 7, and 8 are included in the assay kit as controls for these specific loci probes. Furthermore, CEP1 is unreliable to verify the copy number of the tested genes located on other chromosomes because it only represents chromosome 1. However, we designed the probe panel of the in situ DNA chip solely on the pattern of genomic signatures in primary lung cancer, as defined by our previous comprehensive microarray-based comparative genomic hybridizations. Our chip allows a panel of 16 probes, including eight centromeric probes for enumerating chromosomes, which can more precisely examine the ploidy status (i.e., chromosomal aneuploidy) of the cell than can the LAVysion kit, which only detects one chromosome. More importantly, the in situ DNA chip can detect eight different cancer-specific genetic changes rather than only the three chromosomal loci and has internal control probes, which further permit specific assessment of the cancer-specific genomic aberrations. Therefore, it not surprising that the sensitivity of the chip assay is not only higher than that of cytologic analysis but also markedly higher than that of four-color LAVysion IM-FISH in the detection of cancer cells.

Our technique differs from an approach that divides a single slide into 24 subregions and involves 24 dual-color FISH experiments performed in parallel. First of all, the latter procedure requires a sufficient supply of specimen material and so is particularly difficult when clinical samples are limited in number. Furthermore, the technique requires sophisticated image-capture analysis to ensure that the correct area from the 24 subregions is selected for counting the correct probes on one slide. Our technique also differs from a comparative genomic hybridization-based BAC/PAC microarray in which the hybridization is performed on large DNA fragments immobilized on a matrix. An array consisting of well-defined genomic clones such as BAC or PAC provides complete coverage of some chromosomal arms (e.g., 1p, 3p and 5p), which are frequently altered in a variety of cancers. However, like other throughput array techniques, such an array cannot test samples in situ. In addition, this microarray provides very low resolution for detecting genomic changes of targets, given that BAC or PAC DNA fragments are approximately 100 to 200 kb in length.

Finally, the in situ DNA chip strategy has potential as a rapid and reliable screening method for detecting numeric chromosomal aberrations and tumor-specific genetic changes in nonmitotic cells from a variety of pathologic samples, thus easily determining ploidy, intratumoral heterogeneity, minor focal clones or the clonal evolution of disease, and minimal residual disease. Although we only developed a lung cancer-specific chip in this study, our strategy may also have broad and flexible applicability in the risk assessment, early diagnosis, and staging of other types of cancer, especially when combined with other appropriate chromosomal probes or tumor-specific genes. An automated counting system for signals of the in situ genomic chip will enhance the efficiency of the assay with high-throughput automation.

TABLE 4
Panel of Eight Probes and Their Corresponding Chromosomal Centromeric
Probes Arrayed on the in Situ DNA Chip

| | Probe Name | | |
|---|---|---|---|
| | and | DNA | |
| Square | Chromosomal | Sequence | |
| (element) | Location | Type | Fluorochrome |
| A | CEP1, | Alpha | Texas red |
| | chromosome 1 | satellite | (Qbiogene) |
| | centromere | DNA | |
| | ENO1, | BAC DNA | Alexa Fluor |
| | 1p136.23 | | 555 (yellow) |
| | CEP9, | Alpha | Aqua (Vysis) |
| | chromosome 9 | satellite | |
| | centromere | DNA | |
| | p16, 9p21.3 | LD-DOP- | Alexa Fluor |
| | | PCR DNA | 514 (green) |
| B | CEP3, | Alpha | Texas red |
| | chromosome 3 | satellite | (Qbiogene) |
| | centromere | DNA | |
| | GC20, 3p22.1 | LD-DOP- | Alexa Fluor |
| | | PCR DNA | 555 (yellow) |
| | CEP11, | Alpha | Aqua (Vysis) |
| | chromosome | satellite | |
| | 11 centromere | DNA | |
| | FGF4, 11q13, 3 | LD-DOP- | Alexa Fluor |
| | | PCR DNA | 514 (green) |
| C | CEP8, | Alpha | Texas red |
| | chromosome 8 | satellite | (Qbiogene) |
| | centromere | DNA | |
| | YWHAZ, | BAC DNA | Alexa Fluor |
| | 8q23.1 | | 555 (yellow) |
| | CEP10, | Alpha | Aqua (Vysis) |
| | chromosome | satellite | |
| | 10 centromere | DNA | |
| | SP-A, 10q22.3 | BAC DNA | Alexa Fluor |
| | | | 514 (green) |
| D | CEP7, | Alpha | Texas red |
| | chromosome 7 | satellite | (Qbiogene) |
| | centromere | DNA | |
| | COL1A2, | BAC DNA | Alexa Fluor |
| | 7q21.3 | | 555 (yellow) |
| | CEP15, | Alpha | Aqua (Vysis) |
| | chromosome 15 | satellite | |
| | centromere | DNA | |
| | ANXA2, | BAC DNA | Alexa Fluor |
| | 15q22.2 | | 514 (green) |

s in the cells from the sample in A (original magnification ×600).

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims.

### REFERENCES

1. Minna J D, Roth J A, Gazdar A F. Focus on lung cancer. Cancer Cell 2002. 1: 49-52.
2. Mountain C. T. Revision in the international system for staging of lung cancer. Chest 1997. 111: 1710-1717.
3. Feng Jiang, Nancy P. Caraway, Margaret R. Spitz, Wei Zhang, Ruth L. Katz. Genomic Signatures in Primary Non Small Cell Carcinomas of Lung by Using Comparative Genomic Hybridization Analysis of cDNA microarrays. Neoplaisa, 2004. 6. 623-35.
4. Jiang F, and Kat R L. Expression of GC20, a candidate tumor suppressor gene, is frequently lost in lung cancer. Proc Am Assoc Cancer Res 2002. 43:754.
5. Jiang F, Caraway N P, Katz R L. Prognostic value of pulmonary-associated A1 gene (SFTPA1) deletion in tobacco-related lung cancer. Proc Am Assoc Cancer Res 2002. 43:222.
6. Wistuba I I, Lam S, Behrens C, Virmani A K, Fong K M, Srivastava S, Minna J D, Gazdar A F. Molecular damage in the bronchial epithelium of current and former smokers. J Natl Cancer Inst. 1997 17; 89(18):1366-73.
7. Zabarovsky E R, Lerman M I, Minna J D. Tumor suppressor genes on chromosome 3p involved in the pathogenesis of lung and other cancers. Oncogene. 2002 October 7; 21(45):6915-35.
8. Sasaki S, Kitagawa Y, Sekido Y, Minna J D, Kuwano H, Yokota J, Kohno T. Molecular processes of chromosome 9p21 deletions in human cancers. Oncogene. 2003 June 12; 22(24):3792-8.
9. Hirsch F R, Franklin W A, Gazdar A F, Bunn P A Jr. Early detection of lung cancer: clinical perspectives of recent advances in biology and radiology. Clin Cancer Res. 2001 January; 7(1):5-22.
10. Sidransky D. Emerging molecular markers of cancer. Nat Rev Cancer. 2002 2.21 0-9.
11. Wong M P, Fung L F, Wang E, Chow W S, Chiu S W, Lam W K, Ho K K, Ma E S, Wan T S, Chung L P. Chromosomal aberrations of primary lung adenocarcinomas in nonsmokers. Cancer. 2003 1; 97(5):1263-70.

12. Balsara B R, Testa J R. Chromosomal imbalances in human lung cancer. Oncogene. 2002 October 7; 21(45):6877-83.
13. Goeze A, Schluns K, Wolf G, Thasler Z, Petersen S, Petersen I. Chromosomal imbalances of primary and metastatic lung adenocarcinomas. J Pathol. 2002 196(1):8-16.
14. Nakamura H, Saji H, Ogata A, Hosaka M, Hagiwara M, Saijo T, Kawasaki N, Kato H. cDNA microarray analysis of gene expression in pathologic Stage IA nonsmall cell lung carcinomas. Cancer. 2003 97(11):2798-805.
15. An Q, Pacyna-Gengelbach M, Schluns K, Deutschmann N, Guo S, Gao Y, Zhang J, Cheng S, Petersen I. Identification of differentially expressed genes in immortalized human bronchial epithelial cell line as a model for in vitro study of lung carcinogenesis. Int J Cancer. 2003 103(2):194-204.
16. Heighway J, Knapp T, Boyce L, Brennand S, Field J K, Betticher D C, Donovan M, Lasek A, Rickert P. Expression profiling of primary non-small cell lung cancer for target identification. Oncogene. 2002 21(50):7749-63.
17. Virtanen C, Ishikawa Y, Honjoh D, Kimura M, Shimane M, Miyoshi T, Nomura H, Jones M H. Integrated classification of lung tumors and cell lines by expression profiling. Proc Natl Acad Sci U S A. 2002 17; 99(19):12357-62.
18. Fujii T, Dracheva T, Player A, Chacko S, Clifford R, Strausberg R L, Buetow K, Azumi N, Travis W D, Jen J. A preliminary transcriptome map of non-small cell lung cancer. Cancer Res. 2002; 62(12):3340-6.

19. Nacht M, Dracheva T, Gao Y, Fujii T, Chen Y, Player A, Akmaev V, Cook B, Dufault M, Zhang M, Zhang W, Guo M, Curran J, Han S, Sidransky D, Buetow K, Madden S L, Jen J. Molecular characteristics of non-small cell lung cancer. Proc Natl Acad Sci U S A. 2001 December 18;98(26):15203-8.
20. Tan D, Li Q, Deeb G, Ramnath N, Slocum H K, Brooks J, Cheney R, Wiseman S, Anderson T, Loewen G. Thyroid transcription factor-1 expression prevalence and its clinical implications in non-small cell lung cancer: A high-throughput tissue microarray and immunohistochemistry study. Hum Pathol. 2003 34(6):597-604.
21. Gabrielson E, Berg K, Anbazhagan R. Functional genomics, gene arrays, and the future of pathology. Mod Pathol. 2001 14(12):1294-9.
22. Hyman E, Kauraniemi P, Hautaniemi S, Wolf M, Mousses S, Rozenblum E, Ringner M, Sauter G, Monni O, Elkahloun A, Kallioniemi O P, Kallioniemi A. Impact of DNA amplification on gene expression patterns in breast cancer. Cancer Res. 2002 62(21):6240-5.
23. Pollack J R, Sorlie T, Perou C M, Rees C A, Jeffrey S S, Lonning P E, Tibshirani R, Botstein D, Borresen-Dale A L, Brown P O. Microarray analysis reveals a major direct role of DNA copy number alteration in the transcriptional program of human breast tumors. Proc Natl Acad Sci U S A. 2002 99(20):12963-8.
24. DiFrancesco L M, Murthy S K, Luider J, Demetrick D J. Laser capture microdissection-guided FISH and flow cytometric cell cycle analysis of purified nuclei from paraffin sections. Mod Pathol. 2000 13:705-11.
25. Levsky J M, Singer R H. FISH: past, present and future. J Cell Sci. 2003 116:2833-8.
26. Jiang F, Katz R L. Use of FISH as a powerful diagnostic tool in cytology. Diagn Mol Pathol. 2002 11(1):47-57.
27. Santos Romeo M, Sokolova I A, Morrison L E, Zeng C, Baron A E, Hirsch F R, Miller Y E, Franklin W A, Varella-Garcia M. Chromosomal abnormalities in non-small cell lung carcinomas and in bronchial epithelia of high-risk smokers detected by multi-target interphase fluorescence in situ hybridization. J Mol Diagn. 2003 2:103-12.
28. Barkan G A, Caraway N C, Jiang F, Katz R L. The potential use of FISH to identify predictive markers in early-stage lung carcinomas. Cancer Cytopath. 2004. December 16.
29. Braakhuis B J, Tabor M P, Kummer J A, Leemans C R, Brakenhoff R H. A genetic explanation of Slaughter's concept of field cancerization: evidence and clinical implications. Cancer Res. 2003 April 15;63(8):1727-30.

30. Jeanmart M, Lantuejoul S, Fievet F, Moro D, Sturm N, Brambilla C, Brambilla E. Value of immunohistochemical markers in preinvasive bronchial lesions in risk assessment of lung cancer. Clin Cancer Res. 2003 June; 9(6):2195-203.
31. Belinsky S A, Palmisano W A, Gilliland F D, Crooks L A, Divine K K, Winters S A, Grimes M J, Harms H J, Tellez C S, Smith T M, Moots P P, Lechner J F, Stidley C A, Crowell R E. Aberrant promoter methylation in bronchial epithelium and sputum from current and former smokers. Cancer Res. 2002 April 15;62(8):2370-7.
32. Ayoub J, Jean-Francois R, Cormier Y, Meyer D, Ying Y, Major P, Desjardins C, Bradley W E. Placebo-controlled trial of 13-cis-retinoic acid activity on retinoic acid receptor-beta expression in a population at high risk: implications for chemoprevention of lung cancer. J Clin Oncol 1999. 17: 3546-52.
33. Soria J C, Rodriguez M, Liu D D, Lee J J, Hong W K, Mao L. Aberrant promoter methylation of multiple genes in bronchial brush samples from former cigarette smokers. Cancer Res. 2002 Jan 15;62(2):351-5.
34. Fortina P, Surrey S, Kricka L J. Molecular diagnostics: hurdles for clinical implementation. Trends Mol Med. 2002 8: 264-6.
35. Netten H, Young I T, van Vliet L J, Tanke H J, Vroljik H, Sloos W C. FISH and chips: automation of fluorescent dot counting in interphase cell nuclei. Cytometry. 1997 May 1;28(1):1-10.
36. Kagan J, Liu J, Stein J D, Wagner S S, Babkowski R, Grossman B H, Katz R L. Cluster of allele losses within a 2.5 cM region of chromosome 10 in high-grade invasive bladder cancer. Oncogene 1998 19 :909-13.
37. Jiang F, Desper R, Kallioniemi O P, Sauter G, Mihatsch M J, Moch H. Construction of evolutionary tree models for renal cell carcinoma from comparative genomic hybridization data. Cancer Res 2000 15; 60: 6503.
38. Richter J, Jiang F, Gorog J P, Gasser T C, Moch H, Mihatsch M J, Sauter G. Marked genetic differences between stage pTa and stage pT1 papillary bladder cancer detected by CGH. Cancer Res 1997 15:2860.
39. Jiang F, Richter J, Gasser T, Sauter G, Mihatsch M J, Moch H. Chromosomal imbalances in papillary renal cell carcinoma: genetic differences between histological subtypes. Am J Pathol 1998 153:1467.
40. Desper R, Jiang F, Kallioniemi O P, Moch H, Papadimitriou C H, Schaffer A A. Distance-based reconstruction of tree models for oncogenesis. Comput Biol 2000;7(6):789-803.
41. Dellas A, Torhorst J, Jiang F, Proffitt J, Schultheiss E, Holzgreve W, Sauter G, Mihatsch M J, Moch H. Prognostic value of genomic alterations in invasive cervical squamous cell carcinoma of clinical stage IB detected by comparative genomic hybridization. Cancer Res 1999. 59:3475-9.
42. Sauter G, Gasser T C, Moch H, Richter J, Jiang F, Albrecht R, Novotny H, Wagner U, Bubendorf L, Mihatsch MJ. DNA aberrations in urinary bladder cancer detected by flow cytometry and FISH. Urol Res 1997;26: 31-37.
43. Wistuba I I, Behrens C, Virmani A K, Mele G, Milchgrub S, Girard L, Fondon J W, Garner H R, McKay B, Latif F, Lerman M I, Lam S, Gazdar A F, Minna J D. High resolution chromosome 3p allelotyping of human lung cancer and preneoplastic/preinvasive bronchial epithelium reveals multiple, discontinuous sites of 3p allele loss and three regions of frequent breakpoints. Cancer Res 2000.60:1949-60.
44. Dow R, Hendley J, Pirkmaier A, Musgrove E A, Germain D. Retinoic acid-mediated growth arrest requires ubiquitylation and degradation of the F-box protein Skp2. Biol Chem 2001 10.
45. Feng Jiang, Nancy P. Caraway, Margaret R. Spitz, Wei Zhang, Ruth L. Katz. RNA Silencing of S-Phase Kinase-Interacting Protein 2 (Skp2) Induce Apoptosis and Inhibit Centrosome Amplification in Lung Cancer. Oncogene. 2005, in press.
46. Krishnamurthy S, Katz R L, Shumate A, Strohlein K, Khanna A, Tucker S L, Raijman I, Lahoti S. DNA image analysis combined with routine cytology improves diagnostic sensitivity of common bile duct brushing. Cancer. 2001 June 25;93(3):229-35.
47. Katz R L, Sinkre P A, Zhang H H, Kidd L, Johnston D. Clinical significance of negative and equivocal urinary bladder cytology alone and in combination with DNA image analysis and cystoscopy. Cancer. 1997 December 25;81(6):354-64.
48. de la Roza G L, Hopkovitz A, Caraway N P, Kidd L, Dinney C P, Johnston D, Katz RL.DNA image analysis of urinary cytology: prediction of recurrent transitional cell carcinoma. Mod Pathol. 1996 May; 9(5):571-8.
49. Guidozzi F, Szumel R C, Ball J H, Johnston D A, Katz R L, Kidd L. Image analysis of cellular DNA content in peritoneal fluid of patients with ovarian tumors of low malignant potential and invasive epithelial ovarian cancer. Gynecol Oncol. 1996 May; 61 (2):204-9.
50. Fischler D F, Wongbunnate S, Johnston D A, Katz R L. DNA content by image analysis. An accurate discriminator of malignancy in pericardial effusions. Anal Quant Cytol Histol. 1994 June; 16(3):167-73.
51. Wojcik E M, Katz R L, Johnston D A, Sembera D, el-Naggar A.Comparative analysis of DNA ploidy and proliferative index in fine needle aspirates of non-Hodgkin's lymphomas by image analysis and flow cytometry. Anal Quant Cytol Histol. 1993 June; 15(3):151-7.
52. Ruifrok A C, Katz R L, Johnston D A. Comparison of quantification of histochemical staining by hue-saturation-intensity (HSI) transformation and color-deconvolution. Appl Immunohistochem Mol Morphol. 2003 March; 11(1):85-91.
53. Wang J, Hu L, Hamilton S R, Coombes K R, Zhang W. RNA amplification strategies for cDNA microarray experiments. Biotechniques. 2003 February; 34(2):394-400.
54. Hu L, Wang J, Baggerly K, Wang H, Fuller G N, Hamilton S R, Coombes K R, Zhang W. Obtaining reliable information from minute amounts of RNA using cDNA microarrays. BMC Genomics. 2002 June 21;3(1):16.
55. Taylor E, Cogdell D, Coombes K, Hu L, Ramdas L, Tabor A, Hamilton S, Zhang W. Sequence verification as quality-control step for production of cDNA microarrays. Biotechniques. 2001 July; 31(1):62-5. ).
56. Jiang F, Lin F, Price R, Gu J, Medeiros L J, Zhang H Z, Xie S S, Caraway N P, Katz R L. Rapid detection of IgH/BCL2 rearrangement in follicular lymphoma by interphase fluorescence in situ hybridization with bacterial artificial chromosome probes. J Mol Diagn. 2002 August; 4(3):144-9.
57. Fuller C E, Wang H, Zhang W, Fuller G N, Perry A. High-throughput molecular profiling of high-grade astrocytomas: the utility of fluorescence in situ hybridization on tissue microarrays (TMA-FISH). J Neuropathol Exp Neurol. 2002 61(12):1078-84.
58. Korbling M, Katz R L, Khanna A, Ruifrok A C, Rondon G, Albitar M, Champlin RE, Estrov Z. Hepatocytes and epithelial cells of donor origin in recipients of peripheral-blood stem cells. N Engl J Med. 2002 346:738-46.
59. Wang H, Wang H, Zhang W, Fuller G N. Tissue microarrays: applications in neuropathology research, diagnosis, and education. Brain Pathol. 2002 12(1):95-107.
60. van de Corput M P, Dirks R W, van Gijlswijk R P, van de Rijke F M, Raap A K. Fluorescence in situ hybridization using horseradish peroxidase-labeled oligodeoxynucleotides and tyramide signal amplification for sensitive DNA and mRNA detection. Histochem Cell Biol. 1998 October; 110(4):431-7.

What is claimed is:

1. An in situ genomic DNA array for detection of a disease with genetic aberrations, said DNA array comprising;
a substrate; and
a plurality of genomic DNA probes immobilized to said substrate;
wherein said plurality of genomic DNA probes are selected to identify a genetic signature of said disease and are capable of interphase multiple fluorescence in situ hybridization with a tissue or cell sample.

2. The in situ genomic DNA array of claim 1, wherein said disease is a genetic-related disease.

3. The in situ genomic DNA array of claim 1, wherein said disease is cancer.

4. The in situ genomic DNA array of claim 3, wherein said cancer is lung cancer.

5. The in situ genomic DNA array of claim 1, wherein said substrate is a glass coverslip.

6. The in situ genomic DNA array of claim 1, wherein said plurality of genomic DNA probes comprise gene-specific probes associated with a genetic signature of said disease.

7. The in situ genomic DNA array of claim 6, wherein said plurality of genomic DNA probes further comprise chromosomal centromeric probes that correspond to said gene-specific probes associated with said genetic signature of said disease.

8. The in situ genomic DNA array of claim 1, wherein said genomic DNA probes comprise probes for genes selected from the group consisting of FCN3, MACF 1, CRABP, SNRPE, JTB, Cks1, TNA, GNAI2, SH3BP, GC20, POLR, PLOD, GPX3, DUSP, CD74, CCT5, SKP2, SFTPC, YWHAZ, LAPTM, P16, ELAVL, ACTA1, SFTPA, ARHGEF, MADH6, NME2, RPL38, EPB41L, USP14, RPS16, RPS21, TGFBR, GNB2L, FGF4, MAGED, HYAL2, hTERT, GPC3, and RPL37.

9. The in situ genomic DNA array of claim 1, wherein said genomic DNA probes comprise (1) probes for ENO1, p16, GC20, FGF4, YWHAZ, SP-A, COL1A2, and ANXA2, and (2) chromosomal centromeric probes CEP1, CEP3, CEP7, CEP8, CEP9, CEP10, CEP11, and CEP15.

10. The in situ genomic DNA array of claim 1, wherein said genomic DNA probes are labeled with fluorescence labels.

11. The in situ genomic DNA array of claim 10, wherein said genomic DNA probes are labeled with four different fluorescence labels.

12. A method for detecting a disease with genetic aberrations, said method comprising:
incubating a tissue or cell sample with an in situ genomic DNA array comprising a plurality of genomic DNA probes selected to identify a genetic signature of said disease and capable of interphase multiple fluorescence in situ hybridization with said tissue or cell sample;
detecting a signal from said in situ genomic DNA array; and
producing a diagnosis based on the signal detected from said in situ genomic DNA array.

13. The method of claim 12, further comprising the step of identifying genomic DNA probes specific for a genetic signature of said disease with a tissue microarray.

14. The method of claim 12, wherein said disease is lung cancer and said plurality of genomic DNA probes comprise probes for genes selected from the group consisting of FCN3, MACF1, CRABP, SNRPE, JTB, Cks1, TNA, GNA12, SH3BP, GC20, POLR, PLOD, GPX3, DUSP, CD74, CCT5, SKP2, SFTPC, YWHAZ, LAPTM, P16, ELAVL, ACTAI, SFTPA, ARHGEF, MADH6, NME2, RPL38, EPB41L, USP14, RPS16, RPS21, TGFBR, GNB2L, FGF4, MAGED, HYAL2, hTERT, GPC3, and RPL37.

15. The method of claim 12, wherein said plurality of genomic DNA probes comprise (1) probes for ENO1, p16, GC20, FGF4, YWHAZ, SP-A, COL1A2, and ANXA2, and (2) chromosomal centromeric probes CEP1, CEP3, CEP7, CEP8, CEP9, CEP10, CEP11, and CEP15.

16. The method of claim 12, wherein said tissue or cell sample is selected from the group consisting of sputum, oral brushing and biopsy samples.

17. The method of claim 12, wherein each of the plurality of genomic DNA probes is labeled with four different fluorescence labels.

18. A kit comprising:
at least one genetic probe, wherein the genetic probe comprises a nucleotide sequence specific to a gene having a genetic aberration in a diseased state,
a chromosomal centromeric probe consisting essentially of a nucleotide sequence of the chromosome centromere corresponding to the gene,
wherein said genetic probe and said chromosomal centromeric probe each further comprise a fluorescent label, and
a suitable container.

19. A kit comprising:
at least four genetic probes, wherein each of the genetic probes comprise a nucleotide sequence specific to a first, second, third, and fourth gene, each having a genetic aberration in a diseased state,
a chromosomal centromeric probe, each consisting essentially of a nucleotide sequence of the chromosome centromere corresponding separately to the first, second, third, and fourth gene,
wherein said genetic probes and said chromosomal centromeric probes each further comprise a fluorescent label, and
a suitable container.
